SEQUENCE LISTING (10 sequences)

SEQ ID NO: 1 - DNA, 578 bp, Homo sapiens
  CDS (155)..(418): Gene LBFL302, Clone BC4
cggacgcgtgggttcgaacgttcggactgaggtttttctgcctgaagaagcgtcatacggaccggattgttttcgctggcccagtgtccccggagcttgtgtgcgatacagagagcacctcggaagctgaggcagctggtacttgacagagaggatggcgctgtcgaccatagtctcccagaggaagcagataaagcggaaggctccccgtggctttctaaagcgagtcttcaagcgaaagaagcctcaacttcgtctggagaaaagtggtgacttattggtccatctgaactgtttactgtttgttcatcgattagcagaagagtccaggacaaacgcttgtgcgagtaaatgtagagtcattaacaaggagcatgtactggccgcagcaaaggtaattctaaagaagagcagaggttagaagtcaaagaacatattcttgaaagttatgatgcattcttttgggtggtaacagatcataaagacattttttacacatcagttaatatgggattattaaatattggctataaaaaaaaaaaaaaaaaaaaaaaaaaaaaaaaaaaaaaaaaaaaaaa

SEQ ID NO: 2 - protein, 88 aa, Homo sapiens
MALSTIVSQRKQIKRKAPRGFLKRVFKRKKPQLRLEKSGDLLVHLNCLLFVHRLAEESRTNACASKCRVINKEHVLAAAKVILKKSRG

SEQ ID NO: 3 - DNA, 531 bp, Homo sapiens
  CDS (139)..(402): Gene LBFL302, Clone BC7
cccacgcgtccggaggtttttctgcctgaagaagcgtcatacggaccggattgttttcgctggcccagtgtccccggagcttgtgtgcgatacagagagcacctcggaagctgaggcagctggtacttgacagagaggatggcgctgtcgaccatagtctcccagaggaagcagataaagcggaaggctccccgtggctttctaaagcgagtcttcaagctaaagaagcctcaacttcgtctggagaaaagtggtgacttattggtccatctgaactgtttactgtttgttcatcgattagcagaagagtccaggacaaacgcttgtgcgagtaaatgtagagtcattaacaaggagcatgtactggccgcagcaaaggtaattctaaagaagagcagaggttagaagtcaaagaacatattcttgaaagttatgatgcattcttttgggtggtaacagatcataaagacattttttacacatcagttaatatgggattattaaatattggatataaaaaaaaaaaaaaaa

SEQ ID NO: 4 - protein, 88 aa, Homo sapiens
MALSTIVSQRKQIKRKAPRGFLKRVFKLKKPQLRLEKSGDLLVHLNCLLFVHRLAEESRTNACASKCRVINKEHVLAAAKVILKKSRG

SEQ ID NO: 5 - DNA, 2067 bp, Homo sapiens
  CDS (32)..(1384): Clone GE6
cccgggctgccaggcgcccagctgtgcccagatggatgggacagagacccggcagcggaggctggacagctgtggcaagccaggggagctggggcttcctcaccccctcagcacaggaggactccctgtagcctcagaagatggagctctcagggcccctgagagccaaagcgtgacccccaagccactggagactgagcctagcagggagaccgcctggtccataggccttcaggtgaccgtgcccttcatgtttgcaggcctgggactgtcctgggccggcatgcttctggactatttccaggccaacactggacaaattgatgacccccaggagcagcacagagtcatcagcagcaacctggccctcatccaggtgcaggccactgtcgtggggctcttggctgctgtggctgcgctgctgttgggcgtggtgtctcgagaggaagtggatgtcgccaaggtggagttgctgtgtgccagcagtgtcctcactgccttccttgcagcctttgccctgggggtgctgatggtctgtatagtgattggtgctcgaaagctcggggtcaacccagacaacattgccacgcccattgcagccagcctgggagacctcatcacactgtccattctggctttggttagcagcttcttctacagacacaaagatagtcggtatctgacgccgctggtctgcctcagctttgcggctctgaccccagtgtgggtcctcattgccaagcagagcccacccatcgtgaagatcctgaagtttggctggttcccaatcatcctggccatggtcatcagcagtttcggaggactcatcttgagcaaaaccgtttctaaacagcagtacaaaggcatggcgatatttacccccgtcatatgtggtgttggtggcaatctggtggccattcagaccagccgaatctcaacctacctgcacatgtggagtgcacctggcgtcctgcccctccagatgaagaaattctggcccaacccgtgttctactttctgcacgtcagaaatcaattccatgtcagctcgagtcctgctcttgctggtggtcccaggccatctgattttcttctacatcatctacctggtggagggtcagtcagtcataaacagccagacctttgtggtgctctacctgctggcaggcctgatccaggtgacaatcctgctgtacctggcagaagtgatggttcggctgacttggcaccaggccctggatcctgacaaccactgcatcccctaccttacagggctgggggacctgctcggtactggcctcctggcactctgctttttcactgactggctactgaagagcaaggcagagctgggtggcatctcagaactggcatctggacctccctaactgggccccgctggtcccatttgctcattagaatttcctctcacatcagtgggatacagaattcagtttctcccttgccaggtccttgggatggttgacccctgcctctgcagtagccttttgtgagtctgctaaggtagctctcacacacctcggctctggggttgatacctgagcctgcaatagagccctgaaatcaagagcatggcttgagtgtgtgaatatgatgtgtgcacatgcttaatgagcgtgcaagtgtgcacacgtttgtggagaggagggtgttctggcctgagaaggtaaagaagaggcatgtccagtatgctttgcagggtgtgtttgctcttttccatgcccatgcaacccagattggggtggagcaggaaggagctcttttctgttcccaagcctcagaactcttgagctgtggcttacttgctgtcttcaccaggttcaagctccgtgggccacactgctgctgtgccaagaaggtgtacagcctccccaggatggggcctcatacaacccttcatctgcactcaacatttaatcgtgtccttgctgtctttttattttcctttttgtttgttagcaaaaacctctatttagatttcaataatcagagaagtgtaaaataaaacagattatattgtaaaaaaaaaaaaaaaaaaaaaaaaaaa

SEQ ID NO: 6 - protein, 451 aa, Homo sapiens
MDGTETRQRRLDSCGKPGELGLPHPLSTGGLPVASEDGALRAPESQSVTPKPLETEPSRETAWSIGLQVTVPFMFAGLGLSWAGMLLDYFQANTGQIDDPQEQHRVISSNLALIQVQATVVGLLAAVAALLLGVVSREEVDVAKVELLCASSVLTAFLAAFALGVLMVCIVIGARKLGVNPDNIATPIAASLGDLITLSILALVSSFFYRHKDSRYLTPLVCLSFAALTPVWVLIAKQSPPIVKILKFGWFPIILAMVISSFGGLILSKTVSKQQYKGMAIFTPVICGVGGNLVAIQTSRISTYLHMWSAPGVLPLQMKKFWPNPCSTFCTSEINSMSARVLLLLVVPGHLIFFYIIYLVEGQSVINSQTFVVLYLLAGLIQVTILLYLAEVMVRLTWHQALDPDNHCIPYLTGLGDLLGTGLLALCFFTDWLLKSKAELGGISELASGPP

SEQ ID NO: 7 - DNA, 2178 bp, Homo sapiens
  CDS (41)..(1501): Clone MB5
gggaggggacccgggctgccaggcgcccagctgtgcccagatggatgggacagagacccggcagcggaggctggacagctgtggcaagccaggggagctggggcttcctcaccccctcagcacaggaggactccctgtagcctcagaagatggagctctcagggcccctgagagccaaagcgtgacccccaagccactggagactgagcctagcagggagaccgcctggtccataggccttcaggtgaccgtgcccttcatgtttgcaggcctgggactgtcctgggccggcatgcttctggactatttccagcactggcctgtgtttgtggaggtgaaagaccttttgacattggtgccgcccctggtgggcctgaaggggaacctggagatgacactggcatccagactctccacagctgccaacactggacaaattgatgacccccaggagcagcacagagtcatcagcagcaacctggccctcatccaggtgcaggccactgtcgtggggctcttggctgctgtggctgcgctgctgttgggcgtggtgtctcgagaggaagtggatgtcgccaaggtggagttgctgtgtgccagcagtgtcctcactgccttccttgcagcctttgccctgggggtgctgatggtctgtatagtgattggtgctcgaaagctcggggtcaacccagacaacattgccacgcccattgcagccagcctgggagacctcatcacactgtccattctggctttggttagcagcttcttctacagacacaaagatagtcggtatctgacgccgctggtctgcctcagctttgcggctctgaccccagtgtgggtcctcattgccaagcagagcccacccatcgtgaagatcctgaagtttggctggttcccaatcatcctggccatggtcatcagcagtttcggaggactcatcttgagcaaaaccgtttctaaacagcagtacaaaggcatggcgatatttacccccgtcatatgtggtgttggtggcaatctggtggccattcagaccagccgaatctcaacctacctgcacatgtggagtgcacctggcgtcctgcccctccagatgaagaaattctggcccaacccgtgttctactttctgcacgtcagaaatcaattccatgtcagctcgagtcctgctcttgctggtggtcccaggccatctgattttcttctacatcatctacctggtggagggtcagtcagtcataaacagccagacctttgtggtgctctacctgctggcaggcctgatccaggtgacaatcctgctgtacctggcagaagtgatggttcggctgacttggcaccaggccctggatcctgacaaccactgcatcccctaccttacagggctgggggacctgctcggtactggcctcctggcactctgctttttcactgactggctactgaagagcaaggcagagctgggtggcatctcagaactggcatctggacctccctaactgggccccgctggtcccatttgctcattagaatttcctctcacatcagtgggatacagaattcagtttctcccttgccaggtccttgggatggttgacccctgcctctgcagtagccttttgtgagtctgctaaggtagctctcacacacctcggctctggggttgatacctgagcctgcaatagagccctgaaatcaagagcatggcttgagtgtgtgaatatgatgtgtgcacatgcttaatgagcgtgcaagtgtgcacacgtttgtggagaggagggtgttctggcctgagaaggtaaagaagaggcatgtccagtatgctttgcagggtgtgtttgctcttttccatgcccatgcaacccagattggggtggagcaggaaggagctcttttctgttcccaagcctcagaactcttgagctgtggcttacttgctgtcttcaccaggttcaagctccgtgggccacactgctgctgtgccaagaaggtgtacagcctccccaggatggggcctcatacaacccttcatctgcactcaacatttaatcgtgtccttgctgtctttttattttcctttttgtttgttagcaaaaacctctatttagatttcaataatcagagaagtgtaaaataaaacagattatattgtacttgaaaaaaaaaaaaaaaa

SEQ ID NO: 8 - protein, 487 aa, Homo sapiens
MDGTETRQRRLDSCGKPGELGLPHPLSTGGLPVASEDGALRAPESQSVTPKPLETEPSRETAWSIGLQVTVPFMFAGLGLSWAGMLLDYFQHWPVFVEVKDLLTLVPPLVGLKGNLEMTLASRLSTAANTGQIDDPQEQHRVISSNLALIQVQATVVGLLAAVAALLLGVVSREEVDVAKVELLCASSVLTAFLAAFALGVLMVCIVIGARKLGVNPDNIATPIAASLGDLITLSILALVSSFFYRHKDSRYLTPLVCLSFAALTPVWVLIAKQSPPIVKILKFGWFPIILAMVISSFGGLILSKTVSKQQYKGMAIFTPVICGVGGNLVAIQTSRISTYLHMWSAPGVLPLQMKKFWPNPCSTFCTSEINSMSARVLLLLVVPGHLIFFYIIYLVEGQSVINSQTFVVLYLLAGLIQVTILLYLAEVMVRLTWHQALDPDNHCIPYLTGLGDLLGTGLLALCFFTDWLLKSKAELGGISELASGPP

SEQ ID NO: 9 - DNA, 1616 bp, Homo sapiens
  CDS (31)..(1551): Clone IE4
ccgggctgccaggcgcccagctgtgcccagatggatgggacagagacccggcagcggaggctggacagctgtggcaagccaggggagctggggcttcctcaccccctcagcacaggaggactccctgtagcctcagaagatggagctctcagggcccctgagagccaaagcgtgacccccaagccactggagactgagcctagcagggagaccgcctggtccataggccttcaggtgaccgtgcccttcatgtttgcaggcctgggactgtcctgggccggcatgcttctggactatttccagcactggcctgtgtttgtggaggtgaaagaccttttgacattggtgccgcccctggtgggcctgaaggggaacctggagatgacactggcatccagactctccacagctgccaacactggacaaattgatgacccccaggagcagcacagagtcatcagcagcaacctggccctcatccaggtgcaggccactgtcgtggggctcttggctgctgtggctgcgctgctgttgggcgtggtgtctcgagaggaagtggatgtcgccaaggtggagttgctgtgtgccagcagtgtcctcactgccttccttgcagcctttgccctgggggtgctgatggtctgtatagtgattggtgctcgaaagctcggggtcaacccagacaacattgccacgcccattgcagccagcctgggagacctcatcacactgtccattctggctttggttagcagcttcttctacagacacaaagatagtcggtatctgacgccgctggtctgcctcagctttgcggctctgaccccagtgtgggtcctcattgccaagcagagcccacccatcgtgaagatcctgaagtttggctggttcccaatcatcctggccatggtcatcagcagtttcggaggactcatcttgagcaaaaccgtttctaaacagcagtacaaaggcatggcgatatttacccccgtcatatgtggtgttggtggcaatctggtggccattcagaccagccgaatctcaacctacctgcacatgtggagtgcacctggcgtcctgcccctccagatgaagaaattctggcccaacccgtgttctactttctgcacgtcagaaatcaattccatgtcagctcgagtcctgctcttgctggtggtcccaggccatctgattttcttctacatcatctacctggtggagggtcagtcagtcataaacagccagacctttgtggtgctctacctgctggcaggcctgatccaggtgacaatcctgctgtacctggcagaagtgatggttcggctgacttggcaccaggccctggatcctgacaaccactgcatcccctaccttacagggctgggggacctgctcggttcaagctccgtgggccacactgctgctgtgccaagaaggtgtacagcctccccaggatggggcctcatacaacccttcatctgcactcaacatttaatcgtgtccttgctgtctttttattttcctttttgtttgttagcaaaaacctctatttagatttcaataatcagagaagtgtaaaataaaacagattatattgtaaaaaaaaaaaaaaaaaa

SEQ ID NO: 10 - protein, 507 aa, Homo sapiens
MDGTETRQRRLDSCGKPGELGLPHPLSTGGLPVASEDGALRAPESQSVTPKPLETEPSRETAWSIGLQVTVPFMFAGLGLSWAGMLLDYFQHWPVFVEVKDLLTLVPPLVGLKGNLEMTLASRLSTAANTGQIDDPQEQHRVISSNLALIQVQATVVGLLAAVAALLLGVVSREEVDVAKVELLCASSVLTAFLAAFALGVLMVCIVIGARKLGVNPDNIATPIAASLGDLITLSILALVSSFFYRHKDSRYLTPLVCLSFAALTPVWVLIAKQSPPIVKILKFGWFPIILAMVISSFGGLILSKTVSKQQYKGMAIFTPVICGVGGNLVAIQTSRISTYLHMWSAPGVLPLQMKKFWPNPCSTFCTSEINSMSARVLLLLVVPGHLIFFYIIYLVEGQSVINSQTFVVLYLLAGLIQVTILLYLAEVMVRLTWHQALDPDNHCIPYLTGLGDLLGSSSVGHTAAVPRRCTASPGWGLIQPFICTQHLIVSLLSFYFPFCLLAKTSI

(19) United States
(12) Patent Application Publication
(10) Pub. No.: US 2006/0121471 A1
(43) Pub. Date: Jun. 8, 2006

(54) Gene families associated with stomach cancer

(75) Inventors: Sang-Seok Koh, Taejeon (KR); Hyun-Ho Chung, Taejeon (KR); Bog-Man Lee, Taejeon (KR); Si-Young Song, Taejeon (KR); Qing Liu, Gaithersburg, MD (US); Wen Zeng, Gaitherburg, MD (US)

Correspondence Address: BIRCH STEWART KOLASCH & BIRCH, PO BOX 747, FALLS CHURCH, VA, 22040-0747, US

(73) Assignee: L G Life Sciences ltd., Seoul (KR)

(21) Appl. No.: 10/524,258

(22) PCT Filed: Aug. 14, 2003
(86) PCT No.: PCT/KR03/01653

§ 371 Date: Oct. 3, 2005

Related U.S. Application Data
(60) Provisional application No. 60/402,904, filed on Aug. 14, 2002.
(60) Provisional application No. 60/404,408, filed on Aug. 20, 2002.
(60) Provisional application No. 60/405,304, filed on Aug. 23, 2002.
(60) Provisional application No. 60/421,582, filed on Oct. 28, 2002.

(51) Int. Cl. C12Q 1/68; G01N 33/574; C07H 21/04; C12P 21/06; C07K 14/82; C07K 16/30

(52) U.S. Cl. 435/6; 435/7.23; 435/69.1; 435/320.1; 435/325; 530/350; 530/388.8; 536/23.5

(57) ABSTRACT

The invention relates generally to the changes in gene expression in stomach cancer. The invention relates specifically to human gene families that correspond to mRNA species that are differentially expressed in cancerous stomach tissue compared to normal stomach tissue.

### TECHNICAL FIELD

The invention relates generally to the changes in gene expression in stomach tissue from stomach cancer patients compared to normal stomach tissue. The invention specifically relates to human gene families which are differentially expressed in advanced gastric cancers and other malignant neoplasms compared to normal tissue.

### BACKGROUND ART

Stomach Cancer

In the United States, approximately 24,000 new cases of stomach cancer, or gastric cancer, are diagnosed every year. Although the incidence of stomach cancer has declined significantly in the last 60 years, it is still a serious disease caused by factors that remain elusive. Under similar circumstances, some people develop stomach cancer and others do not.

Stomach cancer usually occurs in people over the age of 55 and is twice as common in men as in women. This type of cancer is not prevalent in the United States, but it is much more prevalent in Japan, Korea, Latin America and parts of Eastern Europe, where people eat more foods that are preserved by drying, pickling, smoking or salting. Conversely, consuming fresh fruits and vegetables may protect against this disease.

Stomach cancer can develop in any part of the stomach and spread throughout the stomach and/or to other organs. The cancer may also grow along the stomach wall and spread to the esophagus or small intestine. If the cancer grows through the stomach wall, it can extend to nearby lymph nodes, the liver and the pancreas and the colon. Stomach cancer can spread even farther, to the ovaries, lungs and distant lymph nodes. When stomach cancer metastasizes to another part of the body, these tumor cells are of the same type as those in the original tumor. In other words, metastasized cells in the liver are still stomach tumor cells. Such tumor cells that spread to an ovary, establishing one or more ovarian tumors, are known as Krukenberg tumors and are composed of transformed stomach cells, not ovarian cells.

Because the symptoms of stomach cancer are non-specific, this cancer is difficult to detect in its early stages. Symptoms include indigestion, heartburn, abdominal pain, nausea and vomiting, diarrhea or constipation, loss of appetite, weakness and fatigue, and bleeding which is detected by blood in the stool or by the affected person vomiting blood. Diagnosis is usually performed by x-rays of the upper gastrointestinal tract and esophagus, the x-rays taken after the patient has consumed a liquid barium tracer. Endoscopy of the stomach and esophagus, with a gastroscope, can also be performed. If abnormal tissue is found, it can be biopsied through the gastroscope. Should the biopsy specimen show cancerous cells, surrounding lymph nodes are then biopsied, and surrounding organs, such as the liver and pancreas, are examined via CT scan to determine the extent or stage of the disease. Treatment methods for stomach cancer are similar to those employed in other types of cancer-removal of the affected organ partial or total gastrectomy), possibly with removal of nearby lymph nodes as well, chemotherapy, radiation therapy and immunotherapy (stimulating immune system components that attack cancer cells) (http://cancernet.nci.nih.gov/cancertypes.html). As early stomach cancer causes few symptoms, diagnosis is not usually made before the advanced stages of the disease, where treatments are less effective.

Molecular Changes in Stomach Cancer

Little is known about the molecular changes in stomach cells associated with the development and progression of stomach cancer. Accordingly, there exists a need for the investigation of the changes in gene expression levels, as well as the need for the identification of new molecular markers associated with the development and progression of stomach cancer. Furthermore, if intervention is expected to be successful in halting or slowing the progression of stomach cancer, means of accurately assessing the early manifestations of this disease need to be established. One way to accurately assess the early manifestations of stomach cancer is to identify markers which are uniquely associated with disease progression (see for example Kim et al. (2001), Oncogene 20: 4568-4575). Likewise, the development of therapeutics to prevent or stop the progression of stomach cancer relies on the identification of genes responsible for cancerous transformation and growth in the stomach.

### DISCLOSURE OF THE INVENTION

The present invention is based on the discovery of new gene families that are differentially expressed in advanced gastric cancer (AGC) and other malignant neoplasms compared to normal tissue. The invention includes an isolated nucleic acid molecule comprising SEQ ID NO: 3, 5, 7, 9, 11, 13, 17 or 19; an isolated nucleic acid molecule that encodes the amino acid sequence of SEQ ID NO: 4, 14 or 18; an isolated nucleic acid molecule that encodes a protein that is expressed in stomach cancer and that exhibits at least about 92% nucleotide sequence identity over the entire length of SEQ ID NO: 3 or 17, an isolated nucleic acid molecule that encodes a protein that is expressed in stomach cancer and that exhibits at least about 95% nucleotide sequence identity over the entire length of SEQ ID NO: 13, and an isolated nucleic acid molecule comprising the complement of any of the aforementioned nucleic acid molecules.

The present invention further includes the nucleic acid molecules operably linked to one or more expression control elements, including vectors comprising the isolated nucleic acid molecules. The invention further includes host cells transformed to contain the nucleic acid molecules of the invention and methods for producing a protein comprising the step of culturing a host cell transformed with a nucleic acid molecule of the invention under conditions in which the protein is expressed.

The invention further provides an isolated polypeptide selected from the group consisting of an isolated polypeptide comprising the amino acid sequence of SEQ ID NO: 4, 6, 8, 10, 12, 14 or 18, an isolated polypeptide comprising a fragment of at least 10 amino acids of SEQ ID NO: 6, 8, 10 or 12, an isolated polypeptide comprising conservative amino acid substitutions of SEQ ID NO: 6, 8, 10 or 12 and an isolated polypeptide comprising naturally occurring amino acid sequence variants of SEQ ID NO: 6, 8, 10 or 12. Polypeptides of the invention also include polypeptides with an amino acid sequence having at least about 90% amino acid sequence identity with the sequence set forth in SEQ ID NO: 4, preferably at least about 92-95%, and more preferably at least about 95-98% sequence identity with the sequence set forth in SEQ ID NO: 4. Polypeptides of the invention also include polypeptides with an amino acid sequence having at least about 50%, 60%, 70% or 75% amino acid sequence identity with the sequence set forth in SEQ ID NO: 6, 8, 10 or 12, preferably at least about 80%, more preferably at least about 90-95%, and most preferably at least about 95-98% sequence identity with the sequence set forth in SEQ ID NO: 6, 8, 10 or 12. Polypeptides of the invention also include polypeptides with an amino acid sequence having at least about 95% and at least about 92% amino acid sequence identity with the sequence set forth in SEQ ID NO: 14 and SEQ ID NO: 18, respectively.

The invention further provides an isolated antibody or antigen-binding antibody fragment that specifically binds to a polypeptide of the invention, including monoclonal and polyclonal antibodies.

The invention further provides methods of identifying an agent which modulates the expression of a nucleic acid molecule encoding a protein of the invention, comprising: exposing cells which express the nucleic acid molecule to the agent; and determining whether the agent modulates expression of said nucleic acid molecule, thereby identifying an agent which modulates the expression of a nucleic acid molecule encoding the protein.

The invention further provides methods of identifying an agent which modulates the level of or at least one activity of a protein of the invention, comprising: exposing cells which express the protein to the agent; and determining whether the agent modulates the level of or at least one activity of said protein, thereby identifying an agent which modulates the level of or at least one activity of the protein.

The invention further provides methods of identifying binding partners for a protein of the invention, comprising the steps of exposing said protein to a potential binding partner; and determining if the potential binding partner binds to said protein, thereby identifying binding partners for the protein.

The present invention further provides methods of modulating the expression of a nucleic acid molecule encoding a protein of the invention, comprising the step of administering an effective amount of an agent which modulates the expression of a nucleic acid molecule encoding the protein. The invention also provides methods of modulating at least one activity of a protein of the invention, comprising the step of administering an effective amount of an agent which modulates at least one activity of the protein of the invention.

The present invention further includes non-human transgenic animals modified to contain the nucleic acid molecules of the invention, or non-human transgenic animals modified to contain the mutated nucleic acid molecules such that expression of the encoded polypeptides of the invention is prevented.

The present invention also includes non-human transgenic animals in which all or a portion of a gene comprising all or a portion of SEQ ID NO: 3, 5, 7, 9, 11, 13 or 17 has been knocked out or deleted from the genome of the animal.

The invention further provides methods of diagnosing stomach cancer or other malignant neoplasms, comprising the steps of acquiring a tissue, blood, urine or other sample from a subject and determining the level of expression of a nucleic acid molecule of the invention or polypeptide of the invention.

The invention further includes compositions comprising a diluent and a polypeptide or protein selected from the group consisting of an isolated polypeptide comprising the amino acid sequence of SEQ ID NO: 4, 6, 8, 10, 12, 14 or 18, an isolated polypeptide with an amino acid sequence having at least about 90% amino acid sequence identity with the sequence set forth in SEQ ID NO: 4, preferably at least about 92-95%, and more preferably at least about 95-98% sequence identity with the sequence set forth in SEQ ID NO: 4, an isolated polypeptide comprising a fragment of at least 10 amino acids of SEQ ID NO: 6, 8, 10 or 12, an isolated polypeptide comprising conservative amino acid substitutions of SEQ ID NO: 6, 8, 10 or 12, naturally occurring amino acid sequence variants of SEQ ID NO: 6, 8, 10 or 12, ah isolated polypeptide with an amino acid sequence having at least about 50%, 60%, 70% or 75% amino acid sequence identity with the sequence set forth in SEQ ID NO: 6, 8, 10 or 12, preferably at least about 80%, more preferably at least about 90-95%, and most preferably at least about 95-98% sequence identity with the sequence set forth in SEQ ID NO: 6, 8, 10 or 12, an isolated polypeptide with at least about 95% amino acid sequence identity with the sequence set forth in SEQ ID NO: 14, or an isolated polypeptide with at least about 92% amino acid sequence identity with the sequence set forth in SEQ ID NO: 18.

### BRIEF DESCRIPTION OF THE DRAWINGS

FIG. 1 FIG. 1 is a diagram showing the sequence differences between SEQ ID NO: 1 (clone AD12) and SEQ ID NO: 3 (clone CH4), which are splice variants of the gene designated LBFL301.

FIG. 2 FIG. 2 is a hydrophobicity plot of the protein encoded by the open reading frame of LBFL301, variant AD12 (SEQ ID NO: 2). Analysis was performed according to the methods of Kyte-Doolittle and Goldman et al.

FIG. 3 FIG. 3 is a hydrophobicity plot of the protein encoded by the open reading frame of LBFL301, variant CH4 (SEQ ID NO: 4). Analysis was performed according to the methods of Kyte-Doolittle and Goldman et al.

FIG. 4 FIG. 4 is a hydrophobicity plot of the protein encoded by the longest of the open reading frames of LBFL304 (SEQ ID NO: 6). Analysis was performed according to the methods of Kyte-Doolittle and Goldman et al.

FIG. 5 FIG. 5 is a hydrophobicity plot of the protein encoded by the open reading frame of LBFL305 (SEQ ID NO: 14). Analysis was performed according to the methods of Kyte-Doolittle and Goldman et al.

FIG. 6 FIG. 6 shows the relative alignment positions of the three LBFL306 clones.

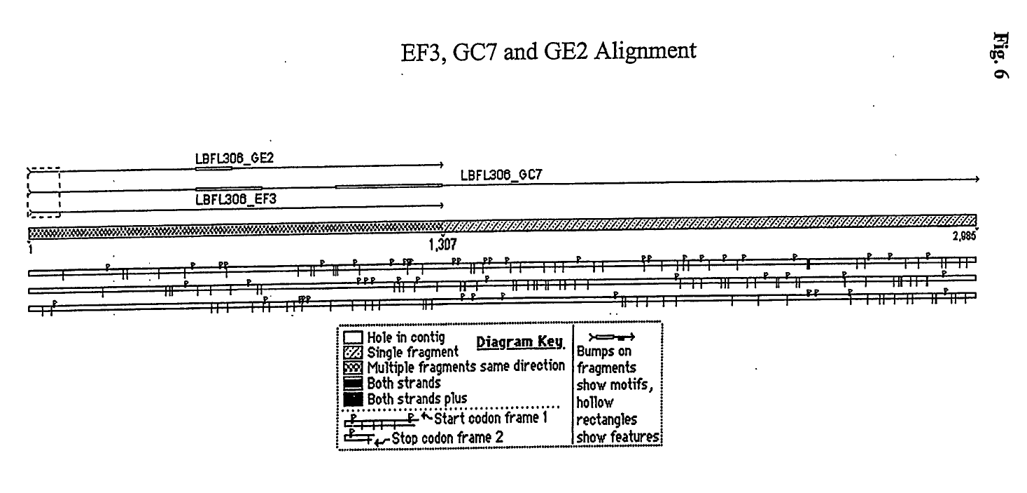

FIG. 7 FIG. 7 is a hydrophobicity plot of the protein encoded by the open reading frame of clone no. LBFL306-EF3 (SEQ ID NO: 18). Analysis was performed according to the methods of Kyte-Doolittle and Goldman et al.

FIG. 8 FIG. 8 is a hydrophobicity plot of the protein encoded by the open reading frame of clone no. LBFL306-GC7 (SEQ ID NO: 20). Analysis was performed according to the methods of Kyte-Doolittle and Goldman et al.

FIG. 9 FIG. 9 is a hydrophobicity plot of the protein encoded by the open reading frame of clone no. LBFL306-GE2 (SEQ ID NO: 22). Analysis was performed according to the methods of Kyte-Doolittle and Goldman et al.

### BEST MODE FOR CARRYING OUT THE INVENTION

I. General Description

The present invention is based in part on the identification of new gene families that are differentially expressed in cancerous human stomach tissue and other malignant neoplasms compared to normal human tissue. These gene families include the human cDNA of SEQ ID NOS: 1, 3, 5, 7, 9, 11, 13, 17, 19 and 21.

The genes and proteins of the invention may be used as diagnostic agents or markers to detect stomach cancer or to monitor the progression of stomach cancer in a sample. They can also serve as a target for agents that modulate gene expression or activity. For example, agents may be identified that modulate biological processes associated with tumor growth, including the hyperplastic process of stomach cancer.

II. Specific Embodiments

A. The Proteins Associated with Stomach Cancer

The present invention provides isolated proteins, allelic variants of the proteins, and conservative amino acid substitutions of the proteins. As used herein, the “protein” or “polypeptide” refers, in part, to a protein that has the human amino acid sequence depicted in SEQ ID NO: 2, 4, 6, 8, 10, 12, 14 or 18. The terms also refer to naturally occurring allelic variants and proteins that have a slightly different amino acid sequence than that specifically recited above. Allelic variants, though possessing a slightly different amino acid sequence than those recited above, will still have the same or similar biological functions associated with these proteins.

As used herein, the families of proteins related to the human amino acid sequence of SEQ ID NO: 2, 4, 6, 8, 10, 12, 14 or 18 include proteins that have been isolated from organisms in addition to humans. The methods used to identify and isolate other members of the family of proteins related to these proteins are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include splice variants and insertion, deletion or conservative amino acid substitution variants of SEQ ID NO: 2, 4, 6, 8, 10, 12, 14 or 18. As used herein, a conservative variant refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein, in certain instances, may be altered without adversely affecting a biological activity. Accordingly, the amino acid sequence can be altered, for example to render the peptide more hydrophobic or hydrophilic, without adversely affecting the biological activities of the protein.

Ordinarily, the allelic variants, the conservative substitution variants, and the members of the protein family encoded by LBFL301, will have an amino acid sequence having at least about 50%, 60%, 70% or 75% amino acid sequence identity with the sequence set forth in SEQ ID NO: 2 or 4, more preferably at least about 80-90%, even more preferably at least about 92-95%, and most preferably at least about 95-98% sequence identity. The allelic variants, the conservative substitution variants, and the members of the protein family encoded by LBFL304, will have an amino acid sequence having at least about 50%, 60%, 70% or 75% amino acid sequence identity with the sequence set forth in SEQ ID NO: 6, 8, 10 or 12, more preferably at least about 80%, even more preferably at least about 90-95%, and most preferably at least about 99 or 99.5% sequence identity. The allelic variants, the conservative substitution variants, and the members of the protein family encoded by LBFL305 or LBFL306, will have an amino acid sequence having at least about 50%, 60%, 70% or 75% amino acid sequence identity with the sequence set forth in SEQ ID NO: 14 or 18, more preferably at least about 80-90%, even more preferably at least about 92-94%, and most preferably at least about 95%, 98% or 99% sequence identity. Identity or homology with respect to such sequences is defined herein as the percentage of amino acid residues in the candidate sequence that are identical with SEQ ID NO: 2, 4, 6, 8, 10, 12, 14 or 18 after aligning the sequences and introducing gaps, if necessary, to achieve the maximum percent homology, and not considering any conservative substitutions as part of the sequence identity (see section B for the relevant parameters). Fusion proteins, or N-terminal, C-terminal or internal extensions, deletions, or insertions into the peptide sequence shall not be construed as affecting homology.

Thus, the proteins of the present invention include molecules having the amino acid sequence disclosed in SEQ ID NO: 2, 4, 6, 8, 10, 12, 14 or 18; fragments thereof having a consecutive sequence of at least about 3, 4, 5, 6, 10, 15, 20, 25, 30, 35 or more amino acid residues of these proteins; amino acid sequence variants wherein one or more amino acid residues has been inserted N- or C-terminal to, or within, the disclosed coding sequence; and amino acid sequence variants of the disclosed sequence, or their fragments as defined above, that have been substituted by at least one residue. Such fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis software such as MacVector (Oxford Molecular).

Contemplated variants further include those containing predetermined mutations by, e.g., homologous recombination, site-directed or PCR mutagenesis, and the corresponding proteins of other animal species, including but not limited to rabbit, mouse, rat, porcine, bovine, ovine, equine and non-human primate species, and the alleles or other naturally occurring variants of the families of proteins (for example, a mouse homolog that shows similarity to the mouse protein corresponding to GenBank Accession No. XM_{—}128002, XM_{—}129365, NM_{—}021420, NM_{—}133971 (DNA sequence) and NP_{—}598732 (protein sequence), all of which are incorporated herein by reference.) Additional variants include derivatives wherein the protein has been covalently modified by substitution, chemical, enzymatic, or other appropriate means with a moiety other than a naturally occurring amino acid (for example a detectable moiety such as an enzyme or radioisotope).

The present invention further provides compositions comprising a protein or polypeptide of the invention and a diluent. Suitable diluents can be aqueous or non-aqueous solvents or a combination thereof, and can comprise additional components, for example water-soluble salts or glycerol, that contribute to the stability, solubility, activity, and/or storage of the protein or polypeptide.

As described below, members of the families of proteins can be used: (1) to identify agents which modulate the level of or at least one activity of the protein, (2) to identify binding partners for the protein, (3) as an antigen to raise polyclonal or monoclonal antibodies, (4) as a therapeutic agent or target and (5) as a diagnostic agent or marker of stomach cancer and other hyperplastic diseases.

B. Nucleic Acid Molecules

The present invention further provides nucleic acid molecules that encode the protein having SEQ ID NO: 2, 4, 6, 8, 10, 12, 14 or 18 and the related proteins herein described, preferably in isolated form. As used herein, “nucleic acid” is defined as RNA or DNA that encodes a protein or peptide as defined above; is complementary to a nucleic acid sequence encoding such peptides; hybridizes to the nucleic acid of SEQ ID NO: 1, 3, 5, 7, 9, 11, 13 or 17 and remains stably bound to it under appropriate stringency conditions; encodes a polypeptide sharing at least about 50%, 60%, 70% or 75%, preferably at least about 80-90%, more preferably at least about 92-95%, and most preferably at least about 95-98% or more identity with the peptide sequence of SEQ ID NO: 2 or 4; exhibits at least 50%, 60%, 70% or 75%, preferably at least about 80-90%, more preferably at least about 92-95%, and even more preferably at least about 95-98% or more nucleotide sequence identity over the open reading frames of SEQ ID NO: 1 or 3; encodes a polypeptide sharing at least about 50%, 60%, 70% or 75%, preferably at least about 80%, more preferably at least about 85%, and most preferably at least about 90%, 95%, 98%, 99%, 99.5% or more identity with the peptide sequence of SEQ ID NO: 6, 8, 10 or 12; exhibits at least 50%, 60%, 70% or 75%, preferably at least about 80%, more preferably at least about 85%, and even more preferably at least about 90%, 95%, 98%, 99%, 99.5% or more nucleotide sequence identity over the open reading frames of SEQ ID NO: 5, 7, 9 or 11; encodes a polypeptide sharing at least about 50%, 60%, 70% or 75%, preferably at least about 80-90%, more preferably at least about 92-94%, and most preferably at least about 95%, 98%, 99% or more identity with the peptide sequence of SEQ ID NO: 14 or 18; or exhibits at least 50%, 60%, 70% or 75%, preferably at least about 80-90%, more preferably at least about 92-94%, and even more preferably at least about 95%, 98%, 99% or more nucleotide sequence identity over the open reading frame of SEQ ID NO: 13 or 17.

The present invention further includes isolated nucleic acid molecules that specifically hybridize to the complement of SEQ ID NO: 1, 3, 5, 7, 9, 11, 13 or 17, particularly molecules that specifically hybridize over the open reading frame. Such molecules that specifically hybridize to the complement of SEQ ID NO: 1, 3, 5, 7, 9, 11, 13 or 17 typically do so under stringent hybridization conditions.

Specifically contemplated are genomic DNA, cDNA, mRNA and antisense molecules, as well as nucleic acids based on alternative backbones or including alternative bases, whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul et al., (1997) Nucleic Acids Res 25:3389-3402, and Karlin et al., (1990) Proc Natl Acad Sci USA 87:2264-2268, both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (1994)

Nature Genetics 6: 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al., (1992) Proc Natl Acad Sci USA 89:10915-10919, fully incorporated by reference), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every wink^{th} position along the query); and gapw-16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal. Preferred molecules are those that hybridize under the above conditions to the complement of SEQ ID NO: 1, 3, 5, 7, 9, 11, 13 or 17 and which encode a functional or full-length protein. Even more preferred hybridizing molecules are those that hybridize under the above conditions to the complement strand of the open reading frame of SEQ ID NO: 1, 3, 5, 7, 9, 11, 13 or 17.

As used herein, a nucleic acid molecule is said to be “isolated” when the nucleic acid molecule is substantially separated from contaminant nucleic acid molecules encoding other polypeptides.

The present invention further provides fragments of the disclosed nucleic acid molecules. As used herein, a fragment of a nucleic acid molecule refers to a small portion of the coding or non-coding sequence. The size of the fragment will be determined by the intended use. For example, if the fragment is chosen so as to encode an active portion of the protein, the fragment will need to be large enough to encode the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming (see the discussion in Section H).

Fragments of the nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphoramidite method of Matteucci et al., ((1981) J Am Chem Soc 103:3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled or fluorescently labeled nucleotides and the like. A skilled artisan can readily employ any such label to obtain labeled variants of the nucleic acid molecules of the invention.

C. Isolation of Other Related Nucleic Acid Molecules

As described above, the identification and characterization of the nucleic acid molecule having SEQ ID NO: 1, 3, 5, 7, 9, 11, 13 or 17 allows a skilled artisan to isolate nucleic acid molecules that encode other members of the protein families in addition to the sequences herein described. Further, the presently disclosed nucleic acid molecules allow a skilled artisan to isolate nucleic acid molecules that encode other members of the families of proteins in addition to the proteins having SEQ ID NO: 2, 4, 6, 8, 10, 12, 14 or 18.

For instance, a skilled artisan can readily use the amino acid sequence of SEQ ID NO: 2, 4, 6, 8, 10, 12, 14 or 18 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gt11 library, to obtain the appropriate coding sequence for other members of the protein families. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Nucleic acid molecules encoding other members of the protein families may also be identified in existing genomic or other sequence information using any available computational method, including but not limited to: PSI-BLAST (Altschul et al., (1997) Nucleic Acids Res 25:3389-3402); PHI-BLAST (Zhang et al., (1998) Nucleic Acids Res 26:3986-3990), 3D-PSSM (Kelly et al., (2000) J Mol Biol 299(2):499-520); and other computational analysis methods (Shi et al., (1999) Biochem Biophys Res Commun 262(1):132-138 and Matsunami et. al., (2000) Nature 404(6778):601-604.

D. rDNA Molecules Containing a Nucleic Acid Molecule

The present invention further provides recombinant DNA molecules (rDNAs) that contain a coding sequence. As used herein, a rDNA molecule is a DNA molecule that has been subjected to molecular manipulation in situ. Methods for generating rDNA molecules are well known in the art, for example, see Sambrook et al., Molecular Cloning—A Laboratory Manual, Third Ed., Cold Spring Harbor Laboratory Press, Cold Spring Harbor, N.Y., 2001. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin, kanamycin, chloramphenicol or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from BioRad Laboratories, (Richmond, Calif.), pPL and pKK223 available from Pharmacia (Piscataway, N.J.).

Expression vectors compatible with eukaryotic cells, preferably those compatible with vertebrate cells, such as stomach cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors. Vectors may be modified to include stomach cell specific promoters if needed.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., (1982) J Mol Anal Genet 1:327-341) Alternatively, the selectable marker can be present on a separate plasmid, and the two vectors are introduced by co-transfection of the host cell, and selected by culturing in the appropriate drug for the selectable marker.

E. Host Cells Containing an Exogenously Supplied Coding Nucleic Acid Molecule

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH/3T3) available from the ATCC as CRL 1658, baby hamster kidney cells (BHK), and the like eukaryotic tissue culture cell lines.

Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed (see, for example, Cohen et al., (1972) Proc Natl Acad Sci USA 69:2110; and Sambrook et al., supra). With regard to transformation of vertebrate cells with vectors containing rDNAs, electroporation, cationic lipid or salt treatment methods are typically employed, see, for example, Graham et al., (1973) Virol 52:456; Wigler et al., (1979) Proc Natl Acad Sci USA 76;1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, (1975) J Mol Biol 98:503 or Berent et al., (1985) Biotech 3:208, or the proteins produced from the cell assayed via an immunological method.

F. Production of Recombinant Proteins Using a rDNA Molecule

The present invention further provides methods for producing a protein of the invention using nucleic acid molecules herein described. In general terms, the production of a recombinant form of a protein typically involves the following steps:

First, a nucleic acid molecule is obtained that encodes a protein of the invention, such as a nucleic acid molecule comprising, consisting essentially of or consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 17, nucleotides 131-862 or 131-859 of SEQ ID NO: 1, nucleotides 174-587 or 174-584 of SEQ ID NO: 3, nucleotides 38-892 or 38-895 of SEQ ID NO: 5, nucleotides 53-892 or 53-895 of SEQ ID NO: 7, nucleotides 65-892 or 65-895 of SEQ ID NO: 9, or nucleotides 92-892 or 92-895 of SEQ ID NO: 11, nucleotides 49-1437 or 49-1434 of SEQ ID NO: 13, or nucleotides 75-575 or 75-572 of SEQ ID NO: 17. If the encoding sequence is uninterrupted by introns, as are these open-reading-frames, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

G. Methods to Identify Binding Partners

Another embodiment of the present invention provides methods for isolating and identifying binding partners of proteins of the invention. In general, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analyzed. To identify and isolate a binding partner, the entire protein, for instance a protein comprising the entire amino acid sequence of SEQ ID NO: 2, 4, 6, 8, 10, 12, 14 or 18 can be used. Alternatively, a fragment of the protein can be used.

As used herein, a cellular extract refers to a preparation or fraction which is made from a lysed or disrupted cell. The preferred source of cellular extracts will be cells derived from human stomach tumors or transformed stomach cells, for instance, biopsy tissue or tissue culture cells from gastric carcinomas. Alternatively, cellular extracts may be prepared from normal tissue or available cell lines, particularly stomach-derived cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al., (1997) Methods Mol Biol 69:171-184 or Sauder et al., (1996) J Gen Virol 77:991-996 or identified through the use of epitope tagged proteins or GST fusion proteins.

Alternatively, the nucleic acid molecules of the invention can be used in a yeast two-hybrid system or other in vivo protein-protein detection system. The yeast two-hybrid system has been used to identify other protein partner pairs and can readily be adapted to employ the nucleic acid molecules herein described.

H. Methods to Identify Agents that Modulate the Expression a Nucleic Acid Encoding the Genes Associated with Stomach Cancer

Another embodiment of the present invention provides methods for identifying agents that modulate the expression of a nucleic acid encoding a protein of the invention such as a protein having the amino acid sequence of SEQ ID NO: 2, 4, 6, 8, 10, 12 or 18, or a Mst1 protein or splice variant of the invention such as a protein having the amino acid sequence of SEQ ID NO: 14. The agents that modulate the expression of the nucleic acid encoding the Mst1 protein or splice variant will have particular use in the treatment of stomach cancer. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, cell lines that contain reporter gene fusions between nucleotides from within the open reading frame defined by nucleotides 131-862 of SEQ ID NO: 1, or nucleotides 174-587 of SEQ ID NO: 3, nucleotides 38-895 of SEQ ID NO: 5, nucleotides 53-895 of SEQ ID NO: 7, nucleotides 65-895 of SEQ ID NO: 9, nucleotides 92-895 of SEQ ID NO: 11, nucleotides 49-1437 or 49-1434 of SEQ ID NO: 13, nucleotides 75-575 of SEQ ID NO: 17 and/or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al., (1990) Anal Biochem 188:245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid of the invention.

Additional assay formats may be used to monitor the ability of the agent to modulate the expression of a nucleic acid encoding a protein of the invention, such as the protein having SEQ ID NO: 2, 4, 6, 8, 10, 12, 14 or 18. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., Molecular Cloning—A Laboratory Manual, Third Ed., Cold Spring Harbor Laboratory Press, Cold Spring Harbor, N.Y., 2001.

The preferred cells will be those derived from human stomach tissue, for instance, stomach biopsy tissue or cultured cells from patients with stomach cancer. Cell lines such as ATCC gastric carcinoma cell line Catalogue Nos. NCI-SNU-16, CRL-1863, HTB-103, CRL-1739 and CRL-1864 may be used. Alternatively, other available cells or cell lines may be used.

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., supra, or Ausubel et al., Short Protocols in Molecular Biology, Fourth Ed., John Wiley & Sons, Inc., New York, 1999.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip, porous glass wafer or membrane. The solid support can then be exposed to total cellular RNA or polyA RNA from a sample under conditions in which the affixed sequences will specifically hybridize. Such solid supports and hybridization methods are widely available, for example, those disclosed by Beattie, (1995) WO 95/11755. By examining for the ability of a given probe to specifically hybridize to an RNA sample from an untreated cell population and from a cell population exposed to the agent, agents which up- or down-regulate the expression of a nucleic acid encoding the protein having the sequence of SEQ ID NO: 2, 4, 6, 8, 10, 12, 14 or 18 are identified.

Hybridization for qualitative and quantitative analysis of mRNAs may also be carried out by using a RNase Protection Assay (i.e., RPA, see Ma et al., (1996) Methods 10:273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes, pH 6.4, 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea/polyacrylamide gels for analysis.

In another assay, to identify agents which affect the expression of the instant gene products, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′promoter-containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al., supra).

Cells or cell lines transduced or transfected as outlined above are then contacted with agents under appropriate conditions. For example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

I. Methods to Identify Agents that Modulate the Level or at Least One Activity of the Stomach Cancer Associated Proteins

Another embodiment of the present invention provides methods for identifying agents that modulate the level or at least one activity of a protein of the invention such as the protein having the amino acid sequence of SEQ ID NO: 2, 4, 6, 8, 10, 12 or 18, or of a Mst1 protein or splice variant of the invention such as the protein having the amino acid sequence of SEQ ID NO: 14. Such methods or assays may utilize any means of monitoring or detecting the desired activity.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. (Rockford, Ill.), may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein ((1975) Nature 256:495-497) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies or the polyclonal antisera which contain the immunologically significant (antigen-binding) portion can be used as antagonists, as well as the intact antibodies. Use of immunologically reactive (antigen-binding) antibody fragments, such as the Fab, Fab′, or F(ab′)₂ fragments is often preferable, especially in a therapeutic context, as these fragments are generally less immunogenic than the whole immunoglobulin.

The antibodies or antigen-binding fragments may also be produced, using current technology, by recombinant means. Antibody regions that bind specifically to the desired regions of the protein can also be produced in the context of chimeras with multiple species origin, such as humanized antibodies.

Agents that are assayed in the above method can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant in: Molecular Biology and Biotechnology, Meyers, ed., pp. 659-664, VCH Publishers, Inc., New York, 1995). A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies.

J. Uses for Agents that Modulate the Expression or at Least One Activity of the Proteins Associated with Stomach Cancer

As provided in the Examples, the proteins and nucleic acids of the invention, such as the proteins having the amino acid sequence of SEQ ID NO: 2, 4, 6, 8, 10, 12 or 18, and the Mst1 or Mst1 splice variant proteins and nucleic acids of the invention, such as the proteins having the amino acid sequence of SEQ ID NO: 14 are differentially expressed in cancerous stomach tissue. Agents that up- or down-regulate or modulate the expression of the protein or at least one activity of the protein, such as agonists or antagonists, of may be used to modulate biological and pathologic processes associated with the protein's function and activity.

For example, two types of drugs have been shown to act through Mst1 (e.g., GenBank Accession No. NM_{—}006282, the nucleic acid and protein sequences for which are given as SEQ ID NOS: 15 and 16, respectively), a gene related to SEQ ID NOS: 13 and 14. Firstly, it has been shown that bisphophonates, drugs that are used to treat osteoporosis and other bone diseases, act directly on the osteoclast to induce caspase cleavage of Mst1 during apoptosis. Secondly, cytotrienin A is an antitumor drug that is used to treat leukemia, breast cancer and lung cancer (U.S. Pat. No. 6,251,885). Cytotrienin A has been shown to activate Mst1 during cytotrienin A-induced apoptosis (Watabe et al., (2000) J Biol Chem 275:8766-8771).

As used herein, a subject can be any mammal, so long as the mammal is in need of modulation of a pathological or biological process mediated by a protein of the invention. The term “mammal” is defined as an individual belonging to the class Mammalia. The invention is particularly useful in the treatment of human subjects.

Pathological processes refer to a category of biological processes which produce a deleterious effect. For example, expression of a protein of the invention may be associated with stomach cell growth or hyperplasia. As used herein, an agent is said to modulate a pathological process when the agent reduces the degree or severity of the process. For instance, stomach cancer may be prevented or disease progression modulated by the administration of agents which up- or down-regulate or modulate in some way the expression or at least one activity of a protein of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of a protein of the invention. While individual needs vary, determination of optimal ranges of effective amounts of each component is within the skill of the art. Typical dosages comprise 0.1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadininistered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro.

K. Transgenic Animals

Transgenic animals containing mutant, knock-out or modified genes corresponding to the cDNA sequence of SEQ ID NO: 1, 3, 5, 7, 9, 11, 13 or 17, or the open reading frame encoding the polypeptide sequence of SEQ ID NO: 2, 4, 6, 8, 10, 12, 14 or 18 or fragments thereof having a consecutive sequence of at least about 3, 4, 5, 6, 10, 15, 20, 25, 30, 35 or more amino acid residues, are also included in the invention. Transgenic animals are genetically modified animals into which recombinant, exogenous or cloned genetic material has been experimentally transferred. Such genetic material is often referred to as a “transgene.” The nucleic acid sequence of the transgene, in this case a form of SEQ ID NO: 1, 3, 5, 7, 9, 11, 13 or 17 may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

In some embodiments, transgenic animals in which all or a portion of a gene comprising SEQ ID NO: 1, 3, 5, 7, 9, 11, 13 or 17 is deleted may be constructed. In those cases where the gene corresponding to SEQ ID NO: 1, 3, 5, 7, 9, 11, 13 or 17 contains one or more introns, the entire gene—all exons, introns and the regulatory sequences—may be deleted. Alternatively, less than the entire gene may be deleted. For example, a single exon and/or intron may be deleted, so as to create an animal expressing a modified version of a protein of the invention.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al., (1993) Hypertension 22:630-633; Brenin et al., (1997) Surg Oncol 6:99-110; Recombinant Gene Expression Protocols (Methods in Molecular Biology, Vol. 62), Tuan, ed., Humana Press, Totowa, N.J., 1997).

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al., (1996) Genetics 143:1753-1760); or, are capable of generating a fully human antibody response (McCarthy (1997) Lancet 349:405).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al., (1997) Mol Reprod Dev 46:515-526; Houdebine, (1995) Reprod Nutr Dev 35:609-617; Petters (1994) Reprod Fertil Dev 6:643-645; Schnieke et al., (1997) Science 278:2130-2133; and Amoah, (1997) J Animal Science 75:578-585).

The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307.

L. Diagnostic Methods

As the genes and proteins of the invention are differentially expressed in cancerous stomach tissue and in other malignant neoplasms compared to non-cancerous tissues of the same type, the genes and proteins of the invention may be used to diagnose or monitor such cancers or to track disease progression. One means of diagnosing cancer, including stomach cancer, using the nucleic acid molecules or proteins of the invention involves obtaining tissue from living subjects, such as biopsy specimens.

The use of molecular biological tools has become routine in forensic technology. For example, nucleic acid probes comprising all or at least part of the sequence of SEQ ID NO: 1, 3, 5, 7, 9, 11, 13 or 17 may be used to determine the expression of a nucleic acid molecule in forensic/pathology specimens. Further, nucleic acid assays may be carried out by any means of conducting a transcriptional profiling analysis. In addition to nucleic acid analysis, forensic methods of the invention may target the proteins of the invention, particularly a protein comprising SEQ ID NO: 2, 4, 6, 8, 10, 12, 14, 16, 18 or 20, to determine up- or down-regulation of the genes (Shiverick et al., (1975) Biochim Biophys Acta 393:124-133).

Methods of the invention may involve treatment of tissues with collagenases or other proteases to make the tissue amenable to cell lysis (Semenov et al., (1987) Biull Eksp Biol Med 104:113-116). Further, it is possible to obtain biopsy samples from different regions of the stomach for analysis.

Assays to detect nucleic acid or protein molecules of the invention may be in any available format. Typical assays for nucleic acid molecules include hybridization or PCR based formats. Typical assays for the detection of proteins, polypeptides or peptides of the invention include the use of antibody probes in any available format such as in situ binding assays, etc. (see Harlow & Lane, Antibodies—A Laboratory Manual, Cold Spring Harbor Laboratory Press, Cold Spring Harbor, N.Y., 1988. In preferred embodiments, assays are carried-out with appropriate controls.

The above methods may also be used in other diagnostic protocols, including protocols and methods to detect disease states in other tissues or organs, for example in tissues in which expression of a nucleic acid molecule of the invention is detected.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1a

Identification of Differentially Expressed mRNA in Advanced Gastric Carcinoma

Materials and Methods

Patient tissue samples were derived from five Korean patients, aged 47 to 68, including four men and one woman, who had been diagnosed with advanced gastric cancer. For each patient, tissue was obtained from two areas of the stomach, from a stomach tumor and from a cancer-free area, to produce a set of biopsy samples. Histological analysis of each of the tissue samples was performed, and samples were segregated into either non-cancerous or cancerous categories.

With minor modifications, the sample preparation protocol followed the Affymetrix GeneChip Expression Analysis Manual. Frozen tissue was first ground to powder using the Spex Certiprep 6800 Freezer Mill. Total RNA was then extracted using Trizol (Life Technologies). The total RNA yield for each sample (average tissue weight of 300 mg) was 200-500 μg. Next, mRNA was isolated using the Oligotex mRNA Midi kit (Qiagen). Since the mRNA was eluted in a final volume of 400 μl, an ethanol precipitation step was required to bring the concentration to 1 μg/μl. Using 1-5 μg of mRNA, double stranded cDNA was created using the SuperScript Choice system (Gibco-BRL). First strand cDNA synthesis was primed with a T7-(dT₂₄) oligonucleotide. The cDNA was then phenol-chloroform extracted and ethanol precipitated to a final concentration of 1 μg/μl

From 2 μg of cDNA, cRNA was synthesized according to standard procedures. To biotin label the cRNA, nucleotides Bio-11-CTP and Bio-16-UTP (Enzo Diagnostics) were added to the reaction. After a 37° C. incubation for six hours, the labeled cRNA was cleaned up according to the RNeasy Mini kit protocol (Qiagen). The cRNA was then fragmented (5× fragmentation buffer: 200 mM Tris-Acetate (pH 8.1), 500 mM KOAc, 150 mM MgOAc) for thirty-five minutes at 94° C.

55 μg of fragmented cRNA was hybridized on the Affymetrix Human Genome U95 and U133 set of arrays for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Following hybridization and scanning, the microarray images were analyzed for quality control, looking for major chip defects or abnormalities in hybridization signal. After all chips passed QC, the data was analyzed using Affymetrix Microarray Suite (v4.0), and LIMS (v1.5) for U95 or Affymetrix Microarray Suite (v5.0), and LIMS (v3.0) for U133.

Differential expression of genes between the cancerous and non-cancerous liver samples was determined by using Affymetrix human GeneChip sets, U95 and U133, with the following statistical methods. (1) For each gene, Affymetrix GeneChip average difference values for U95 were determined by Affymetrix Microarray Suite (v4.0), which also made “Absent” (=not detected), “Tresent” (=detected) or “Marginal” (=not clearly Absent or Present) calls for each GeneChip element. Signal values for U133 were determined by Affymetrix Microarray Suite (v5.0), which also made Absent, Present or Marginal calls. (2) Using the criteria of at least 10% present call in both cancerous and non-cancerous liver samples and at least 40% present call in either cancerous or non-cancerous liver sample groups, a gene set was selected for further analysis. (3) Based on the average difference values of U95 data, the gene set was split into two groups, a high expression group and low expression group. The high expression group contained genes with average difference values greater than or equal to 5 in both cancerous and non-cancerous samples. The remainder of the genes were included in the low expression group. The average difference values were transformed to a logarithmic scale for the high expression group, but were not changed for the low expression group. For U133 data, all signal values were transformed to a logarithmic scale regardless of expression level. (4) The Analysis of Variance (ANOVA) method was used for data analysis (Steel et al., Principles and Procedures of Statistics: A Biometrical Approach, Third Ed., McGraw-Hill, 1997). Prior to the final analysis, a leave-one-out approach is used for outlier detection. One sample at a time was left out of the ANOVA analysis to determine whether or not omitting a specific sample from the analysis had any significant effect on the final result. If so, that particular sample was excluded from the final analysis. After outlier detection, a list of genes that are differentially expressed with a p-value of less than or equal to 0.05 was generated by ANOVA. Data from Affymetrix GeneChip U133 chip set was analyzed with a similar procedure. (5) Two additional criteria were used to reduce the number of genes in the gene list generated from U95. Firstly, geometric mean values were compared between the non-cancerous control group samples and the carcinoma disease group samples to obtain a set of genes showing at least 2.0-fold increases or decreases in expression level. Secondly, the ratio of the fold-change value and the p-value had to be 400 or greater.

Results and Analyses

a) LBFL301 Gene Family:

Analysis of the chip data showed that the expression of the marker LBFL301 was significantly up-regulated (13.75-fold; p=0.0172) in gastric carcinoma samples compared to samples from normal stomach tissue. These data indicate that up-regulation of LBFL301 may be diagnostic for stomach cancer.

The expression level of LBFL301 (SEQ ID NO: 1 or 3) can be measured by chip sequence fragment nos. 48774_at and 225681_at on Affymetrix GeneChips® U95 and U133, respectively. The expression levels of 48774_at and 225681_at in various malignant neoplasms, compared to normal control tissues, are shown in Table 1a, where the fold-change and the direction of the change (up- or down-regulation) are also indicated. A fold-change greater than 1.5 was considered to be significant.

TABLE 1a

| Affy ID | Tissue | Disease | Morphology | Fold Change | Dir | T-Stat |
|---|---|---|---|---|---|---|
| 48774_at | BONES, NOS | MALIGNANT NEOPLASM OF BONE, NOS | GIANT CELL TUMOR OF BONE, NOS | 5.7 | Up | 3.5 |
| 225681_at | BREAST, NOS | MALIGNANT NEOPLASM OF FEMALE BREAST, NOS | INFILTRATING DUCT CARCINOMA | 3.4 | Up | 12.1 |
| 225681_at | | | INFILTRATING DUCT AND LOBULAR CARCINOMA | 3.3 | Up | 3.3 |
| 225681_at | | | INFILTRATING LOBULAR CARCINOMA | 2.8 | Up | 5.3 |
| 225681_at | CERVIX, NOS | MALIGNANT NEOPLASM OF UTERINE CERVIX | SQUAMOUS CELL CARCINOMA, NOS | 3.6 | Up | 3.4 |
| 225681_at | COLON, NOS | MALIGNANT NEOPLASM OF COLON, NOS | MUCINOUS ADENOCARCINOMA | 12.7 | Up | 2.5 |
| 225681_at | | | ADENOCARCINOMA, NOS | 6.9 | Up | 7.9 |
| 225681_at | ENDOMETRIUM, NOS | MALIGNANT NEOPLASM OF ENDOMETRIUM | MULLERIAN MIXED TUMOR | 11.0 | Up | 5.5 |
| 225681_at | | | ADENOCARCINOMA, NOS | 2.2 | Up | 3.7 |
| 225681_at | ESOPHAGUS, NOS | MALIGNANT NEOPLASM OF ESOPHAGUS, NOS | ADENOCARCINOMA, NOS | 7.2 | Up | 3.6 |
| 225681_at | KIDNEY, NOS | MALIGNANT NEOPLASM OF KIDNEY, NOS | CLEAR CELL ADENOCARCINOMA, NOS | 9.8 | Up | 5.4 |
| 225681_at | | | WILMS' TUMOR | 7.2 | Up | 3.4 |
| 225681_at | LARYNX, NOS | MALIGNANT NEOPLASM OF LARYNX, NOS | SQUAMOUS CELL CARCINOMA, NOS | 6.6 | Up | 4.3 |
| 225681_at | LIVER, NOS | SECONDARY MALIGNANT NEOPLASM OF LIVER, NOS | ADENOCARCINOMA, NOS | 12.4 | Up | 3.5 |
| 225681_at | LUNG, NOS | MALIGNANT NEOPLASM OF LUNG, NOS | SQUAMOUS CELL CARCINOMA, NOS | 5.4 | Up | 5.4 |
| 225681_at | | | ADENOCARCINOMA, NOS | 4.4 | Up | 5.1 |
| 225681_at | LYMPH NODE, NOS | SECONDARY MALIGNANT NEOPLASM OF LYMPH NODE, NOS | SQUAMOUS CELL CARCINOMA, NOS | 4.1 | Up | 2.7 |
| 225681_at | | MALIGNANT NEOPLASM OF LYMPHOID AND HISTIOCYTIC | MALIGNANT LYMPHOMA, NOS | −4.3 | Down | −3.2 |
| | | TISSUE, NOS | | | | |
| 225681_at | OMENTUM, NOS | MALIGNANT NEOPLASM OF THE OMENTUM | PAPILLARY SEROUS ADENOCARCINOMA | 7.2 | Up | 4.1 |
| 225681_at | | SECONDARY MALIGNANT NEOPLASM OF THE OMENTUM | PAPILLARY SEROUS ADENOCARCINOMA | 4.8 | Up | 4.3 |
| 225681_at | | | MULLERIAN MIXED TUMOR | 4.7 | Up | 11.0 |
| 225681_at | OVARY, NOS | MALIGNANT NEOPLASM OF OVARY | ADENOCARCINOMA, NOS | 13.8 | Up | 2.6 |
| 225681_at | | | SEROUS CYSTADENOCARCINOMA, NOS | 10.7 | Up | 2.8 |
| 225681_at | | NEOPLASM OF UNCERTAIN BEHAVIOR OF OVARY | STRUMA OVARII, NOS | 9.5 | Up | 6.4 |
| 225681_at | | MALIGNANT NEOPLASM OF OVARY | PAPILLARY SEROUS ADENOCARCINOMA | 7.7 | Up | 3.7 |
| 225681_at | | SECONDARY MALIGNANT NEOPLASM OF OVARY | ADENOCARCINOMA, NOS | 6.9 | Up | 3.7 |
| 225681_at | | NEOPLASM OF UNCERTAIN BEHAVIOR OF OVARY | SEROUS CYSTADENOMA, BORDERLINE | 5.3 | Up | 7.2 |
| | | | MALIGNANCY | | | |
| 225681_at | PANCREAS, NOS | MALIGNANT NEOPLASM OF PANCREAS, NOS | ADENOCARCINOMA, NOS | 7.8 | Up | 9.3 |
| 225681_at | RECTUM, NOS | MALIGNANT NEOPLASM OF RECTUM | ADENOCARCINOMA, NOS | 7.7 | Up | 5.8 |
| 225681_at | SOFT TISSUES, NOS | NEOPLASM OF UNCERTAIN BEHAVIOR OF CONNECTIVE AND | FIBROMATOSIS, NOS | 12.5 | Up | 7.6 |
| | | OTHER SOFT TISSUES, NOS | | | | |
| 225681_at | | SECONDARY MALIGNANT NEOPLASM OF CONNECTIVE AND | SQUAMOUS CELL CARCINOMA, NOS | 5.3 | Up | 3.2 |
| | | OTHER SOFT TISSUES, NOS | | | | |
| 225681_at | | MALIGNANT NEOPLASM OF CONNECTIVE AND OTHER SOFT | FIBROUS HISTIOCYTOMA, MALIGNANT | 3.7 | Up | 3.1 |
| | | TISSUES, NOS | | | | |
| 225681_at | STOMACH, NOS | MALIGNANT NEOPLASM OF STOMACH, NOS | ADENOCARCINOMA, NOS | 3.6 | Up | 3.5 |
| 225681_at | | | SIGNET RING CELL CARCINOMA | 3.5 | Up | 4.5 |
| 225681_at | THYROID GLAND, | MALIGNANT NEOPLASM OF THYROID GLAND | PAPILLARY CARCINOMA, NOS | 4.5 | Up | 4.1 |
| | NOS | | | | | |

Table 2 summarizes the differential expression data collected from experiments using Affymetrix GeneChips by tissue type. The chips were scanned and the data analyzed by the GX Scan algorithm, which is described in related applications 60/331,182, 60/388,745 and 60/390,608, all entitled “An Automated Computer-based Algorithm for Organizing and Mining Gene Expression Data Derived from Biological Samples with Complex Clinical Attributes,” and all of which are herein incorporated by reference in their entirety.

TABLE 2
LBFL301 (U95: 48774_at, U133: 225681_at): Clones AD12 & CH4

| | | 48774_at | 225681_at |
|---|---|---|---|
| | | From U95 data | From U133 data |
| 1. | Bone | UP | — |
| 2. | Breast | UP | UP |
| 3. | Cervix | UP | UP |
| 4. | Colon | UP | UP |
| 5. | Endometrium | UP | UP |
| 6. | Esophagus | UP | UP |
| 7. | Kidney | UP | UP |
| 8. | Larynx | UP | UP |
| 9. | Liver | UP | UP |
| 10. | Lung | UP | UP |
| 11. | Omentum | UP | UP |
| 12. | Ovary | UP | UP |
| 13. | Pancreas | UP | UP |
| 14. | Rectum | UP | UP |
| 15. | Soft tissues | UP | UP |
| 16. | Stomach | UP | UP |
| 17. | Thyroid Gland | UP | UP |

The GeneChip expression results, determined by sample binding to chip sequence fragment no. 48774_at were validated by quantitative RT-PCR using Taqman® assay (Perkin-Elmer). PCR primers designed from the sif sequence of the specific Affymetrix fragment (48774_at) were used in the assay. The target gene in each RNA sample (ten ng of total RNA) was assayed relative to an exogenously spiked reference gene. For this purpose, the tetracycline resistance gene was used as the exogenously added spike. This approach provides the relative expression as measured by cycle threshold (Ct) value of the target mRNA relative to a constant amount of Tet spike Ct values. The sample panel included normal and advanced gastric cancer tissue RNAs that were analyzed on U95 GeneChips. In addition, several new samples that were not analyzed on the GeneChip were used for the expression validations by Quantitative RT-PCR. The Q-RT-PCR data confirms the up-regulation of LBFL301 observed in advanced gastric cancer.

b) LBFL304 Gene Family:

Analysis of the chip data showed that the expression of the marker LBFL304 was significantly up-regulated (3.5-fold, p=2.54×10⁻³ for U95; 6.13-fold, p=2.43×10⁻⁴ for U133) in AGC samples compared to samples from normal stomach tissue. This data indicates that up-regulation of LBFL304 may be diagnostic for stomach cancer.

The expression level of LBFL304 (SEQ ID NO: 5, 7, 9 or 11) can be measured by chip sequence fragment nos. 35832_at on Affymetrix GeneChips® U95 and 212344_at, 212353_at, and 212354_at on Affymetrix GeneChips® U133. The expression levels of 51263_at, 212344_at, 212353_at, and 212354_at in various malignant neoplasms, compared to normal control tissues, are shown in Table 1b, where the fold-change and the direction of the change (up- or down-regulation) are also indicated. A fold-change greater than 1.5 was considered to be significant.

The GeneChip expression results, determined by sample binding to chip sequence fragment no. 35832_at, were validated by quantitative RT-PCR (Q-RT-PCR) using the Taqman® assay (Perkin-Elmer). PCR primers designed from the sequence information file of the specific Affymetrix fragment (35832_at) were used in the assay. The target gene in each RNA sample (10 ng of total RNA) was assayed relative to an exogenously spiked reference gene. For this purpose, the tetracycline resistance gene was used as the exogenously added spike. This approach provides the relative expression as measured by cycle threshold (Ct) value of the target mRNA relative to a constant amount of Tet spike Ct values. The sample panel included normal stomach (Normal) and advanced gastric cancer (AGC) tissue RNAs that were analyzed on U95 GeneChips. In addition, several new samples that were not analyzed on the GeneChip were used for the expression validations by Q-RT-PCR The Q-RT-PCR data confirms the up-regulation of LBFL304 observed in AGC, compared to normal stomach biopsy samples.

TABLE 1
Table 1b

| | | | | Fold | | |
|---|---|---|---|---|---|---|
| Affy ID | Tissue | Disease | Morphology | Change | Dir | T-Stat |
| 212344_at | BONES, NOS | MALIGNANT NEOPLASM OF BONE, | GIANT CELL TUMOR OF BONE, NOS | 6.8 | Up | 6.9 |
| | | NOS | | | | |
| 212353_at | | MALIGNANT NEOPLASM OF BONE, | GIANT CELL TUMOR OF BONE, NOS | 5.5 | Up | 5.7 |
| | | NOS | | | | |
| 212354_at | | MALIGNANT NEOPLASM OF BONE, | GIANT CELL TUMOR OF BONE, NOS | 5.9 | Up | 7.1 |
| | | NOS | | | | |
| 35832_at | | MALIGNANT NEOPLASM OF BONE, | GIANT CELL TUMOR OF BONE, NOS | 10.6 | Up | 5.7 |
| | | NOS | | | | |
| 212344_at | COLON, NOS | MALIGNANT NEOPLASM OF | MUCINOUS ADENOCARCINOMA | 3.7 | Up | 2.8 |
| | | COLON, NOS | | | | |
| 212344_at | | MALIGNANT NEOPLASM OF | ADENOCARCINOMA, NOS | 2.7 | Up | 7.9 |
| | | COLON, NOS | | | | |
| 212353_at | | MALIGNANT NEOPLASM OF | MUCINOUS ADENOCARCINOMA | 5.7 | Up | 3.7 |
| | | COLON, NOS | | | | |
| 212353_at | | MALIGNANT NEOPLASM OF | ADENOCARCINOMA, NOS | 4.1 | Up | 9.0 |
| | | COLON, NOS | | | | |
| 212354_at | | MALIGNANT NEOPLASM OF | MUCINOUS ADENOCARCINOMA | 4.7 | Up | 3.5 |
| | | COLON, NOS | | | | |
| 212354_at | | MALIGNANT NEOPLASM OF | ADENOCARCINOMA, NOS | 3.0 | Up | 8.6 |
| | | COLON, NOS | | | | |
| 35832_at | | MALIGNANT NEOPLASM OF | MUCINOUS ADENOCARCINOMA | 9.4 | Up | 3.8 |
| | | COLON, NOS | | | | |
| 35832_at | | MALIGNANT NEOPLASM OF | ADENOCARCINOMA, NOS | 6.5 | Up | 10.2 |
| | | COLON, NOS | | | | |
| 212344_at | SOFT | MALIGNANT NEOPLASM OF | FIBROUS HISTIOCYTOMA, | 2.7 | Up | 3.2 |
| | TISSUES, | CONNECTIVE AND OTHER SOFT | MALIGNANT | | | |
| | NOS | TISSUES, NOS | | | | |
| 212353_at | | MALIGNANT NEOPLASM OF | FIBROUS HISTIOCYTOMA, | 2.1 | Up | 2.2 |
| | | CONNECTIVE AND OTHER SOFT | MALIGNANT | | | |
| | | TISSUES, NOS | | | | |
| 212353_at | | MALIGNANT NEOPLASM OF | MYXOID LIPOSARCOMA | −2.7 | Down | −2.4 |
| | | CONNECTIVE AND OTHER SOFT | | | | |
| | | TISSUES, NOS | | | | |
| 212354_at | | MALIGNANT NEOPLASM OF | FIBROUS HISTIOCYTOMA, | 2.2 | Up | 2.6 |
| | | CONNECTIVE AND OTHER SOFT | MALIGNANT | | | |
| | | TISSUES, NOS | | | | |
| 35832_at | | MALIGNANT NEOPLASM OF | FIBROUS HISTIOCYTOMA, | 3.5 | Up | 2.6 |
| | | CONNECTIVE AND OTHER SOFT | MALIGNANT | | | |
| | | TISSUES, NOS | | | | |
| 35832_at | | MALIGNANT NEOPLASM OF | LIPOSARCOMA, NOS | 4.8 | Up | 2.6 |
| | | CONNECTIVE AND OTHER SOFT | | | | |
| | | TISSUES, NOS | | | | |
| 212344_at | ENDOMETRIUM, | MALIGNANT NEOPLASM OF | MULLERIAN MIXED TUMOR | 2.4 | Up | 3.3 |
| | NOS | ENDOMETRIUM | | | | |
| 212344_at | | MALIGNANT NEOPLASM OF | ADENOCARCINOMA, NOS | 2.2 | Up | 5.5 |
| | | ENDOMETRIUM | | | | |
| 212353_at | | MALIGNANT NEOPLASM OF | MULLERIAN MIXED TUMOR | 1.9 | Up | 2.9 |
| | | ENDOMETRIUM | | | | |
| 212353_at | | MALIGNANT NEOPLASM OF | ADENOCARCINOMA, NOS | 2.0 | Up | 4.2 |
| | | ENDOMETRIUM | | | | |
| 212354_at | | MALIGNANT NEOPLASM OF | MULLERIAN MIXED TUMOR | 2.0 | Up | 2.7 |
| | | ENDOMETRIUM | | | | |
| 212354_at | | MALIGNANT NEOPLASM OF | ADENOCARCINOMA, NOS | 1.9 | Up | 4.7 |
| | | ENDOMETRIUM | | | | |
| 35832_at | | MALIGNANT NEOPLASM OF | MULLERIAN MIXED TUMOR | 3.0 | Up | 3.9 |
| | | ENDOMETRIUM | | | | |
| 35832_at | | MALIGNANT NEOPLASM OF | ADENOCARCINOMA, NOS | 2.4 | Up | 4.7 |
| | | ENDOMETRIUM | | | | |
| 212344_at | ESOPHAGUS, | MALIGNANT NEOPLASM OF | ADENOCARCINOMA, NOS | 1.7 | Up | 2.3 |
| | NOS | ESOPHAGUS, NOS | | | | |
| 212353_at | | MALIGNANT NEOPLASM OF | ADENOCARCINOMA, NOS | 5.5 | Up | 3.2 |
| | | ESOPHAGUS, NOS | | | | |
| 212354_at | | MALIGNANT NEOPLASM OF | ADENOCARCINOMA, NOS | 2.9 | Up | 3.1 |
| | | ESOPHAGUS, NOS | | | | |
| 35832_at | | MALIGNANT NEOPLASM OF | ADENOCARCINOMA, NOS | 10.7 | Up | 3.2 |
| | | ESOPHAGUS, NOS | | | | |
| 212344_at | BREAST, | MALIGNANT NEOPLASM OF | MUCINOUS ADENOCARCINOMA | 3.3 | Up | 4.8 |
| | NOS | FEMALE BREAST, NOS | | | | |
| 212344_at | | MALIGNANT NEOPLASM OF | INFILTRATING LOBULAR | 1.6 | Up | 2.9 |
| | | FEMALE BREAST, NOS | CARCINOMA | | | |
| 212344_at | | MALIGNANT NEOPLASM OF | INFILTRATING DUCT CARCINOMA | 2.5 | Up | 8.7 |
| | | FEMALE BREAST, NOS | | | | |
| 212344_at | | MALIGNANT NEOPLASM OF | INFILTRATING DUCT AND LOBULAR | 2.6 | Up | 2.8 |
| | | FEMALE BREAST, NOS | CARCINOMA | | | |
| 212353_at | | MALIGNANT NEOPLASM OF | INFILTRATING LOBULAR | 2.8 | Up | 4.7 |
| | | FEMALE BREAST, NOS | CARCINOMA | | | |
| 212353_at | | MALIGNANT NEOPLASM OF | INFILTRATING DUCT CARCINOMA | 3.9 | Up | 13.0 |
| | | FEMALE BREAST, NOS | | | | |
| 212353_at | | MALIGNANT NEOPLASM OF | INFILTRATING DUCT AND LOBULAR | 3.4 | Up | 3.7 |
| | | FEMALE BREAST, NOS | CARCINOMA | | | |
| 212354_at | | MALIGNANT NEOPLASM OF | INFILTRATING LOBULAR | 1.7 | Up | 3.1 |
| | | FEMALE BREAST, NOS | CARCINOMA | | | |
| 212354_at | | MALIGNANT NEOPLASM OF | INFILTRATING DUCT CARCINOMA | 2.6 | Up | 8.5 |
| | | FEMALE BREAST, NOS | | | | |
| 212354_at | | MALIGNANT NEOPLASM OF | INFILTRATING DUCT AND LOBULAR | 2.7 | Up | 2.6 |
| | | FEMALE BREAST, NOS | CARCINOMA | | | |
| 35832_at | | MALIGNANT NEOPLASM OF | MUCINOUS ADENOCARCINOMA | 4.0 | Up | 5.5 |
| | | FEMALE BREAST, NOS | | | | |
| 35832_at | | MALIGNANT NEOPLASM OF | INFILTRATING LOBULAR | 3.2 | Up | 4.4 |
| | | FEMALE BREAST, NOS | CARCINOMA | | | |
| 35832_at | | MALIGNANT NEOPLASM OF | INFILTRATING DUCT CARCINOMA | 4.5 | Up | 12.0 |
| | | FEMALE BREAST, NOS | | | | |
| 35832_at | | MALIGNANT NEOPLASM OF | INFILTRATING DUCT AND LOBULAR | 4.3 | Up | 4.5 |
| | | FEMALE BREAST, NOS | CARCINOMA | | | |
| 212353_at | KIDNEY, NOS | MALIGNANT NEOPLASM OF | CLEAR CELL ADENOCARCINOMA, | 2.8 | Up | 5.5 |
| | | KIDNEY, NOS | NOS | | | |
| 212354_at | | MALIGNANT NEOPLASM OF | CLEAR CELL ADENOCARCINOMA, | 2.4 | Up | 5.3 |
| | | KIDNEY, NOS | NOS | | | |
| 35832_at | | MALIGNANT NEOPLASM OF | CLEAR CELL ADENOCARCINOMA, | 6.1 | Up | 5.6 |
| | | KIDNEY, NOS | NOS | | | |
| 212344_at | LARYNX, | MALIGNANT NEOPLASM OF | SQUAMOUS CELL CARCINOMA, | 3.0 | Up | 3.3 |
| | NOS | LARYNX, NOS | NOS | | | |
| 212353_at | | MALIGNANT NEOPLASM OF | SQUAMOUS CELL CARCINOMA, | 5.1 | Up | 3.8 |
| | | LARYNX, NOS | NOS | | | |
| 212354_at | | MALIGNANT NEOPLASM OF | SQUAMOUS CELL CARCINOMA, | 3.7 | Up | 3.6 |
| | | LARYNX, NOS | NOS | | | |
| 35832_at | | MALIGNANT NEOPLASM OF | SQUAMOUS CELL CARCINOMA, | 7.2 | Up | 3.7 |
| | | LARYNX, NOS | NOS | | | |
| 212344_at | LUNG, NOS | MALIGNANT NEOPLASM OF LUNG, | CARCINOMA, NOS | 4.1 | Up | 3.9 |
| | | NOS | | | | |
| 212344_at | | MALIGNANT NEOPLASM OF LUNG, | SQUAMOUS CELL CARCINOMA, | 3.1 | Up | 5.8 |
| | | NOS | NOS | | | |
| 212344_at | | MALIGNANT NEOPLASM OF LUNG, | LARGE CELL CARCINOMA, NOS | 2.7 | Up | 2.5 |
| | | NOS | | | | |
| 212344_at | | MALIGNANT NEOPLASM OF LUNG, | ADENOCARCINOMA, NOS | 2.8 | Up | 6.2 |
| | | NOS | | | | |
| 212353_at | | MALIGNANT NEOPLASM OF LUNG, | CARCINOMA, NOS | 4.1 | Up | 3.2 |
| | | NOS | | | | |
| 212353_at | | MALIGNANT NEOPLASM OF LUNG, | SQUAMOUS CELL CARCINOMA, | 3.6 | Up | 6.5 |
| | | NOS | NOS | | | |
| 212353_at | | MALIGNANT NEOPLASM OF LUNG, | LARGE CELL CARCINOMA, NOS | 3.8 | Up | 2.7 |
| | | NOS | | | | |
| 212353_at | | MALIGNANT NEOPLASM OF LUNG, | ADENOCARCINOMA, NOS | 3.6 | Up | 6.9 |
| | | NOS | | | | |
| 212354_at | | MALIGNANT NEOPLASM OF LUNG, | CARCINOMA, NOS | 3.5 | Up | 2.9 |
| | | NOS | | | | |
| 212354_at | | MALIGNANT NEOPLASM OF LUNG, | SQUAMOUS CELL CARCINOMA, | 3.1 | Up | 5.6 |
| | | NOS | NOS | | | |
| 212354_at | | MALIGNANT NEOPLASM OF LUNG, | LARGE CELL CARCINOMA, NOS | 2.9 | Up | 2.6 |
| | | NOS | | | | |
| 212354_at | | MALIGNANT NEOPLASM OF LUNG, | ADENOCARCINOMA, NOS | 2.9 | Up | 6.2 |
| | | NOS | | | | |
| 35832_at | | MALIGNANT NEOPLASM OF LUNG, | SQUAMOUS CELL CARCINOMA, | 8.8 | Up | 6.0 |
| | | NOS | NOS | | | |
| 35832_at | | MALIGNANT NEOPLASM OF LUNG, | ADENOCARCINOMA, NOS | 8.9 | Up | 7.2 |
| | | NOS | | | | |
| 212344_at | OVARY, NOS | MALIGNANT NEOPLASM OF OVARY | PAPILLARY SEROUS | 3.0 | Up | 3.9 |
| | | | ADENOCARCINOMA | | | |
| 212353_at | | MALIGNANT NEOPLASM OF OVARY | PAPILLARY SEROUS | 3.6 | Up | 3.9 |
| | | | ADENOCARCINOMA | | | |
| 212354_at | | MALIGNANT NEOPLASM OF OVARY | PAPILLARY SEROUS | 2.6 | Up | 3.4 |
| | | | ADENOCARCINOMA | | | |
| 212344_at | PANCREAS, | MALIGNANT NEOPLASM OF | ADENOCARCINOMA, NOS | 6.9 | Up | 9.0 |
| | NOS | PANCREAS, NOS | | | | |
| 212353_at | | MALIGNANT NEOPLASM OF | ADENOCARCINOMA, NOS | 7.8 | Up | 11.2 |
| | | PANCREAS, NOS | | | | |
| 212354_at | | MALIGNANT NEOPLASM OF | ADENOCARCINOMA, NOS | 8.6 | Up | 11.0 |
| | | PANCREAS, NOS | | | | |
| 35832_at | | MALIGNANT NEOPLASM OF | ADENOCARCINOMA, NOS | 99.8 | Up | 11.1 |
| | | PANCREAS, NOS | | | | |
| 212353_at | PROSTATE, | MALIGNANT NEOPLASM OF | ATYPIA SUSPICIOUS FOR | −2.2 | Down | −4.7 |
| | NOS | PROSTATE | MALIGNANCY | | | |
| 212353_at | | MALIGNANT NEOPLASM OF | ADENOCARCINOMA, NOS | −1.5 | Down | −2.5 |
| | | PROSTATE | | | | |
| 212354_at | | MALIGNANT NEOPLASM OF | ATYPIA SUSPICIOUS FOR | −1.8 | Down | −3.8 |
| | | PROSTATE | MALIGNANCY | | | |
| 212354_at | | MALIGNANT NEOPLASM OF | ADENOCARCINOMA, NOS | −1.7 | Down | −3.8 |
| | | PROSTATE | | | | |
| 35832_at | | MALIGNANT NEOPLASM OF | ATYPIA SUSPICIOUS FOR | −4.3 | Down | −4.4 |
| | | PROSTATE | MALIGNANCY | | | |
| 35832_at | | MALIGNANT NEOPLASM OF | ADENOCARCINOMA, NOS | −1.7 | Down | −2.6 |
| | | PROSTATE | | | | |
| 212344_at | RECTUM, | MALIGNANT NEOPLASM OF | ADENOCARCINOMA, NOS | 3.0 | Up | 6.9 |
| | NOS | RECTUM | | | | |
| 212353_at | | MALIGNANT NEOPLASM OF | ADENOCARCINOMA, NOS | 4.1 | Up | 7.6 |
| | | RECTUM | | | | |
| 212354_at | | MALIGNANT NEOPLASM OF | ADENOCARCINOMA, NOS | 3.0 | Up | 7.2 |
| | | RECTUM | | | | |
| 35832_at | | MALIGNANT NEOPLASM OF | ADENOCARCINOMA, NOS | 5.4 | Up | 8.8 |
| | | RECTUM | | | | |
| 212344_at | SKIN, NOS | MALIGNANT NEOPLASM OF SKIN, | SQUAMOUS CELL CARCINOMA, | 2.8 | Up | 2.4 |
| | | NOS | NOS | | | |
| 212353_at | | MALIGNANT NEOPLASM OF SKIN, | SQUAMOUS CELL CARCINOMA, | 3.5 | Up | 2.4 |
| | | NOS | NOS | | | |
| 35832_at | | MALIGNANT NEOPLASM OF SKIN, | BASAL CELL CARCINOMA, NOS | 3.6 | Up | 2.5 |
| | | NOS | | | | |
| 35832_at | | MALIGNANT NEOPLASM OF SKIN, | SQUAMOUS CELL CARCINOMA, | 5.5 | Up | 3.0 |
| | | NOS | NOS | | | |
| 212344_at | STOMACH, | MALIGNANT NEOPLASM OF | MUCINOUS ADENOCARCINOMA | 7.6 | Up | 3.1 |
| | NOS | STOMACH, NOS | | | | |
| 212344_at | | MALIGNANT NEOPLASM OF | SIGNET RING CELL CARCINOMA | 3.9 | Up | 2.4 |
| | | STOMACH, NOS | | | | |
| 212344_at | | MALIGNANT NEOPLASM OF | ADENOCARCINOMA, NOS | 2.5 | Up | 4.9 |
| | | STOMACH, NOS | | | | |
| 212353_at | | MALIGNANT NEOPLASM OF | MUCINOUS ADENOCARCINOMA | 11.0 | Up | 3.7 |
| | | STOMACH, NOS | | | | |
| 212353_at | | MALIGNANT NEOPLASM OF | SIGNET RING CELL CARCINOMA | 4.9 | Up | 2.9 |
| | | STOMACH, NOS | | | | |
| 212353_at | | MALIGNANT NEOPLASM OF | ADENOCARCINOMA, NOS | 3.4 | Up | 5.6 |
| | | STOMACH, NOS | | | | |
| 212354_at | | MALIGNANT NEOPLASM OF | MUCINOUS ADENOCARCINOMA | 7.5 | Up | 3.2 |
| | | STOMACH, NOS | | | | |
| 212354_at | | MALIGNANT NEOPLASM OF | SIGNET RING CELL CARCINOMA | 3.7 | Up | 2.4 |
| | | STOMACH, NOS | | | | |
| 212354_at | | MALIGNANT NEOPLASM OF | ADENOCARCINOMA, NOS | 2.7 | Up | 5.1 |
| | | STOMACH, NOS | | | | |
| 35832_at | | MALIGNANT NEOPLASM OF | SIGNET RING CELL CARCINOMA | 8.4 | Up | 3.4 |
| | | STOMACH, NOS | | | | |
| 35832_at | | MALIGNANT NEOPLASM OF | ADENOCARCINOMA, NOS | 8.6 | Up | 6.7 |
| | | STOMACH, NOS | | | | |
| 212344_at | TESTIS, NOS | MALIGNANT NEOPLASM OF | MIXED GERM CELL TUMOR | 2.0 | Up | 2.4 |
| | | TESTIS, NOS | | | | |
| 212353_at | | MALIGNANT NEOPLASM OF | MIXED GERM CELL TUMOR | 4.1 | Up | 2.8 |
| | | TESTIS, NOS | | | | |
| 212354_at | | MALIGNANT NEOPLASM OF | MIXED GERM CELL TUMOR | 3.4 | Up | 2.6 |
| | | TESTIS, NOS | | | | |
| 212353_at | OMENTUM, | MALIGNANT NEOPLASM OF THE | PAPILLARY SEROUS | 1.7 | Up | 2.6 |
| | NOS | OMENTUM | ADENOCARCINOMA | | | |
| 35832_at | THYROID | MALIGNANT NEOPLASM OF | PAPILLARY CARCINOMA, NOS | 2.5 | Up | 2.5 |
| | GLAND, NOS | THYROID GLAND | | | | |
| 35832_at | CERVIX, NOS | MALIGNANT NEOPLASM OF | SQUAMOUS CELL CARCINOMA, | 3.5 | Up | 2.3 |
| | | UTERINE CERVIX | NOS | | | |

c) LBFL305 Gene Family:

Analysis of the chip data showed that the expression of the marker LBFL305 was significantly up-regulated (2.2-fold, p=0.0051 using the U95 GeneChip; 2.14-fold, p=0.0109 using the U133 GeneChip) in gastric carcinoma samples compared to samples from normal stomach tissue. These data indicate that up-regulation of LBFL305 may be diagnostic for stomach cancer.

The expression level of LBFL305 (SEQ ID NO: 13) can be measured by chip sequence fragment nos. 53858_at and 225364_at on Affymetrix GeneChips® U95 and U133, respectively. Differential expression data were collected from experiments using Affymetrix GeneChips® by tissue type and were analyzed by the GX Scan algorithm, which is described in related applications 60/331,182, 60/388,745 and 60/390,608, all entitled “An Automated Computer-based Algorithm for Organizing and Mining Gene Expression Data Derived from Biological Samples with Complex Clinical Attributes,” and all of which are herein incorporated by reference in their entirety. The expression levels of 53858_at and 225364_at in various malignant neoplasms, compared to normal control tissues, are shown in Table 1c, where the fold-change and the direction of the change (up- or down-regulation) are also indicated. A fold-change greater than 1.5 was considered to be significant.

TABLE 1c

| Affy ID | Tissue | Disease | Morphology | Fold Change | Dir | T-Stat |
|---|---|---|---|---|---|---|
| 53858_at | BLADDER, NOS | MALIGNANT NEOPLASM OF BLADDER, NOS | TRANSITIONAL CELL CARCINOMA, NOS | 1.691 | Up | 2.377 |
| 53858_at | BREAST, NOS | MALIGNANT NEOPLASM OF FEMALE BREAST, NOS | INFILTRATING DUCT CARCINOMA | 1.538 | Up | 6.824 |
| 53858_at | | MALIGNANT NEOPLASM OF FEMALE BREAST, NOS | INFILTRATING DUCT & LOBULAR CARCINOMA | 1.599 | Up | 2.408 |
| 225364_at | | MALIGNANT NEOPLASM OF FEMALE BREAST, NOS | INFILTRATING DUCT & LOBULAR CARCINOMA | 1.525 | Up | 3.251 |
| 53858_at | CERVIX, NOS | MALIGNANT NEOPLASM OF UTERINE CERVIX | SQUAMOUS CELL CARCINOMA, NOS | 1.637 | Up | 4.056 |
| 53858_at | ENDOMETRIUM, NOS | MALIGNANT NEOPLASM OF ENDOMETRIUM | MULLERIAN MIXED TUMOR | 1.548 | Up | 2.647 |
| 53858_at | ESOPHAGUS, NOS | MALIGNANT NEOPLASM OF ESOPHAGUS, NOS | ADENOCARCINOMA, NOS | 2.079 | Up | 3.145 |
| 225364_at | | MALIGNANT NEOPLASM OF ESOPHAGUS, NOS | ADENOCARCINOMA, NOS | 1.615 | Up | 2.915 |
| 53858_at | KIDNEY, NOS | MALIGNANT NEOPLASM OF KIDNEY, NOS | RENAL CELL CARCINOMA | 1.864 | Up | 6.093 |
| 53858_at | | MALIGNANT NEOPLASM OF KIDNEY, NOS | CLEAR CELL ADENOCARCINOMA, NOS | 2.167 | Up | 6.877 |
| 225364_at | | MALIGNANT NEOPLASM OF KIDNEY, NOS | RENAL CELL CARCINOMA | 1.593 | Up | 5.045 |
| 225364_at | | MALIGNANT NEOPLASM OF KIDNEY, NOS | CLEAR CELL ADENOCARCINOMA, NOS | 1.683 | Up | 7.924 |
| 225364_at | LUNG, NOS | MALIGNANT NEOPLASM OF LUNG, NOS | SMALL CELL CARCINOMA, NOS | −1.79 | Down | −7.071 |
| 53858_at | LYMPH NODE, NOS | HODGKIN'S DISEASE, NOS OF LYMPH NODES OF MULTIPLE SITES | HODGKIN'S DISEASE, NOS | 1.539 | Up | 2.519 |
| 53858_at | | MALIGNANT NEOPLASM OF LYMPHOID AND HISTIOCYTIC | MALIGNANT LYMPHOMA, NOS | 1.635 | Up | 3.243 |
| | | TISSUE, NOS | | | | |
| 53858_at | PANCREAS, NOS | MALIGNANT NEOPLASM OF ISLETS OF LANGERHANS | ISLET CELL CARCINOMA | 3.845 | Up | 7.929 |
| 53858_at | | MALIGNANT NEOPLASM OF PANCREAS, NOS | ADENOCARCINOMA, NOS | 4.171 | Up | 7.165 |
| 225364_at | | MALIGNANT NEOPLASM OF ISLETS OF LANGERHANS | ISLET CELL CARCINOMA | 2.343 | Up | 3.551 |
| 225364_at | | MALIGNANT NEOPLASM OF PANCREAS, NOS | ADENOCARCINOMA, NOS | 2.278 | Up | 10.793 |
| 225364_at | PROSTATE, NOS | MALIGNANT NEOPLASM OF PROSTATE | ATYPIA SUSPICIOUS FOR MALIGNANCY | −1.641 | Down | −4.8 |
| 53858_at | RECTUM, NOS | MALIGNANT NEOPLASM OF RECTUM | ADENOCARCINOMA, NOS | 1.935 | Up | 5.416 |
| 53858_at | SMALL INTESTINE, NOS | MALIGNANT LYMPHOMA, NOS OF UNSPECIFIED, | MALIGNANT LYMPHOMA, NOS | 3.935 | Up | 3.535 |
| | | EXTRANODAL OR SOLID ORGAN SITE | | | | |
| 225364_at | | MALIGNANT LYMPHOMA, NOS OF UNSPECIFIED, | MALIGNANT LYMPHOMA, NOS | 3.024 | Up | 3.816 |
| | | EXTRANODAL OR SOLID ORGAN SITE | | | | |
| 53858_at | STOMACH, NOS | MALIGNANT NEOPLASM OF STOMACH, NOS | SIGNET RING CELL CARCINOMA | 1.507 | Up | 2.46 |
| 53858_at | | MALIGNANT NEOPLASM OF STOMACH, NOS | ADENOCARCINOMA, NOS | 2.076 | Up | 5.928 |
| 225364_at | | MALIGNANT NEOPLASM OF STOMACH, NOS | SIGNET RING CELL CARCINOMA | 1.524 | Up | 3.076 |
| 225364_at | | MALIGNANT NEOPLASM OF STOMACH, NOS | ADENOCARCINOMA, NOS | 1.637 | Up | 6.053 |
| 225364_at | THYROID GLAND, NOS | MALIGNANT LYMPHOMA, NOS OF UNSPECIFIED, | MALIGNANT LYMPHOMA, NOS | 4.371 | Up | 9.429 |
| | | EXTRANODAL OR SOLID ORGAN SITE | | | | |
| 225364_at | | MALIGNANT NEOPLASM OF THYROID GLAND | PAPILLARY CARCINOMA, NOS | 1.634 | Up | 3.22 |
| 53858_at | VULVA, NOS | MALIGNANT NEOPLASM OF VULVA, NOS | SQUAMOUS CELL CARCINOMA, NOS | 1.505 | Up | 2.596 |

The GeneChip expression results, determined by sample binding to chip sequence fragment no. 53858_at were validated by quantitative RT-PCR (Q-RT-PCR) using Taqman® assay (Perkin-Elmer). PCR primers designed from the sequence information file for the specific Affymetrix fragment (53858_at) were used in the assay. The target gene in each RNA sample (ten ng of total RNA) was assayed relative to an exogenously spiked reference gene. For this purpose, the tetracycline resistance gene was used as the exogenously added spike. This approach provides the relative expression as measured by cycle threshold (Ct) value of the target mRNA relative to a constant amount of Tet spike Ct values. The sample panel included normal and advanced gastric cancer tissue RNAs that were analyzed on U95 GeneChips. In addition, several new samples that were not analyzed on the GeneChip were used for the expression validations by Q-RT-PCR. The Q-RT-PCR data confirms the up-regulation of LBFL305 observed in advanced gastric cancer.

d) LBFL306 Gene Family

Analysis of the chip data showed that the expression of the marker LBFL306 was significantly up-regulated (3.27-fold, p=0.00217 using the U133 GeneChip) in gastric carcinoma samples compared to samples from normal stomach tissue. These data indicate that up-regulation of LBFL306 may be diagnostic for stomach cancer.

The expression level of LBFL306 (SEQ ID NO: 17, 19 or 21) can be measured by chip sequence fragment nos. 57861_at and 223251_s_at on Affymetrix GeneChips® U95 and U133, respectively. Differential expression data were collected from experiments using Affymetrix GeneChips® by tissue type and were analyzed by the GX Scan algorithm, which is described in related applications 60/331,182, 60/388,745 and 60/390,608, all entitled “An Automated Computer-based Algorithm for Organizing and Mining Gene Expression Data Derived from Biological Samples with Complex Clinical Attributes,” and all of which are herein incorporated by reference in their entirety. The expression levels of 223251_at in various malignant neoplasms, compared to normal control tissues, are shown in Table 1d, where the fold-change and the direction of the change (up- or down-regulation) are also indicated. A fold-change greater than 1.5 was considered to be significant. The data show that expression of LBFL306 is up-regulated in cancers of the bladder, colon, esophagus, kidney, omentum, pancreas, rectum and soft tissues, in addition to cancer of the stomach, and that expression of this gene family is down-regulated in cancers of the breast, endometrium and small intestine.

The full length cDNA having SEQ ID NO: 17 or 19 or 21 was obtained by using GeneTrapper® cDNA Positive Selection System Kits (Invitrogen). The resulting cDNA was converted to double-stranded plasmid DNA, used to transform E. coli cells (DH10B), and the longest cDNA was screened. After positive selection was confirmed by PCR using gene-specific primers, the cDNA clone was subjected to DNA sequencing.

Analysis by Northern blot was performed to determine the size of the mRNA transcripts that correspond to LBFL306. Northern blots containing total RNAs from various human tissues were used (ClonTech H12), and LBFL306-GE2 (SEQ ID NO: 21) was radioactively labeled by the random primer method and used to probe the blots. The blots were hybridized in Church and Gilbert buffer at 65° C. and washed with 0.1×SSC containing 0.1% SDS at room temperature. The Northern blots show a single transcript for this gene, which is approximately 1.5 kb in size. This corresponds to the size of the insert in full-length clones, which is also approximately 1.5 kb.

The GeneChip expression results, determined by sample binding to chip sequence fragment no. 223251_s_at were validated by quantitative RT-PCR (Q-RT-PCR) using Taqman® assay (Perkin-Elmer). PCR primers designed from the sequence information file for the specific Affymetrix fragment (223251_s_at) were used in the assay. The target gene in each RNA sample (ten ng of total RNA) was assayed relative to an exogenously spiked reference gene. For this purpose, the tetracycline resistance gene was used as the exogenously added spike. This approach provides the relative expression as measured by cycle threshold (Ct) value of the target mRNA relative to a constant amount of Tet spike Ct values. The sample panel included normal and advanced gastric cancer tissue RNAs that were analyzed on U133 GeneChips. In addition, several new samples that were not analyzed on the GeneChip were used for the expression validations by Q-RT-PCR The Q-RT-PCR data conforms the up-regulation of LBFL306 observed in advanced gastric cancer.

TABLE 1d

| Chip position | | | | Fold | | |
|---|---|---|---|---|---|---|
| no. | Tissue | Disease | Morphology | change | Dir | T-Stat |
| 223251_s_at | BLADDER, NOS | MALIGNANT NEOPLASM OF BLADDER, NOS | TRANSITIONAL CELL | 1.649 | Up | 2.572 |
| | | | CARCINOMA, NOS | | | |
| 223251_s_at | BREAST, NOS | MALIGNANT NEOPLASM OF FEMALE | INTRADUCTAL | −1.701 | Down | −5.202 |
| | | BREAST, NOS | CARCINOMA, NOS | | | |
| 223251_s_at | COLON, NOS | MALIGNANT NEOPLASM OF COLON, NOS | ADENOCARCINOMA, NOS | 2.651 | Up | 8.164 |
| 223251_s_at | ENDOMETRIUM, | MALIGNANT NEOPLASM OF ENDOMETRIUM | MULLERIAN MIXED | −1.806 | Down | −2.367 |
| | NOS | | TUMOR | | | |
| 223251_s_at | ESOPHAGUS, | MALIGNANT NEOPLASM OF | ADENOCARCINOMA, NOS | 5.016 | Up | 4.269 |
| | NOS | ESOPHAGUS, NOS | | | | |
| 223251_s_at | KIDNEY, NOS | MALIGNANT NEOPLASM OF KIDNEY, NOS | TRANSITIONAL CELL | 1.755 | Up | 4.563 |
| | | | CARCINOMA, NOS | | | |
| 223251_s_at | | MALIGNANT NEOPLASM OF KIDNEY, NOS | CLEAR CELL | 1.611 | Up | 4.054 |
| | | | ADENOCARCINOMA, NOS | | | |
| 223251_s_at | | MALIGNANT NEOPLASM OF KIDNEY, NOS | WILMS' TUMOR | 4.565 | Up | 3.891 |
| 223251_s_at | OMENTUM, NOS | MALIGNANT NEOPLASM OF THE OMENTUM | PAPILLARY SEROUS | 1.762 | Up | 3.398 |
| | | | ADENOCARCINOMA | | | |
| 223251_s_at | PANCREAS, NOS | MALIGNANT NEOPLASM OF PANCREAS, NOS | ADENOCARCINOMA, NOS | 2.672 | Up | 7.13 |
| 223251_s_at | RECTUM, NOS | MALIGNANT NEOPLASM OF RECTUM | ADENOCARCINOMA, NOS | 2.482 | Up | 6.042 |
| 223251_s_at | SMALL | MALIGNANT NEOPLASM OF SMALL | SARCOMA, NOS | −1.944 | Down | −5.716 |
| | INTESTINE, NOS | INTESTINE, NOS | | | | |
| 223251_s_at | SOFT TISSUES, | MALIGNANT NEOPLASM OF CONNECTIVE | MYXOID LIPOSARCOMA | 1.878 | Up | 3.894 |
| | NOS | AND OTHER SOFT TISSUES, NOS | | | | |
| 223251_s_at | STOMACH, NOS | MALIGNANT NEOPLASM OF STOMACH, NOS | ADENOCARCINOMA, NOS | 2.522 | Up | 5.101 |

### Example 2

Cloning of Full Length human cDNAs (LBFL301, LBFL304, LBFL305 and LBFL306) Corresponding to Differentially Expressed mRNA Species

The full length cDNA having SEQ ID NO: 1, 3, 5, 7, 9, 11, 13, 17, 19 or 21 was obtained by the oligo-pulling method. Briefly, a gene-specific oligo was designed based on the sequence of LBFL301, LBFL304, LBFL305 or LBFL306. The oligo was labeled with biotin and used to hybridize with 2 μg of single strand plasmid DNA (cDNA recombinants) from a fully differentiated stomach adenocarcinoma library (NCI CGAP Gas 4) or a library prepared from Jurkat cells following the procedures of Sambrook et al. The hybridized cDNAs were separated by streptavidin-conjugated beads and eluted by heating. The eluted cDNA was converted to double strand plasmid DNA and used to transform E. coli cells (DH10B) and the longest cDNA was screened. After positive selection was confirmed by PCR using gene-specific primers, the cDNA clone was subjected to DNA sequencing.

The nucleotide sequence of the full-length human cDNAs corresponding to the differentially regulated mRNA detected above is set forth in SEQ ID NOS: 1, 3, 5, 7, 9, 11, 13, 17, 19 and 21. In SEQ ID NO 1, the cDNA comprises 1272 base pairs (1255 base pairs and a polyA tail). In SEQ ID NO 3, the cDNA comprises 1355 base pairs (1334 base pairs and a polyA tail). There are several possible start codons for LBFL304, and they are designated in SEQ ID NOS: 5, 7, 9 and 11. The cDNA in SEQ ID NO: 13 comprises 6405 base paris (6369 base pairs and a poly A tail). The cDNA corresponding to SEQ ID NO: 17 comprises 1299 base pairs (1284 base pairs and a polyA tail). The cDNA corresponding to SEQ ID NO: 19 comprises 2451 base pairs (2435 base pairs and a polyA tail). The cDNA corresponding to SEQ ID NO: 21 comprises 1194 base pairs (1178 base pairs and a polyA tail).

An open reading frame within the cDNA nucleotide sequence of SEQ ID NO: 1, at nucleotides 131-859 (131-862 including the stop codon), encodes a protein of 243 amino acids. The amino acid sequence corresponding to a predicted protein encoded by SEQ ID NO: 1 is set forth in SEQ ID NO: 2.

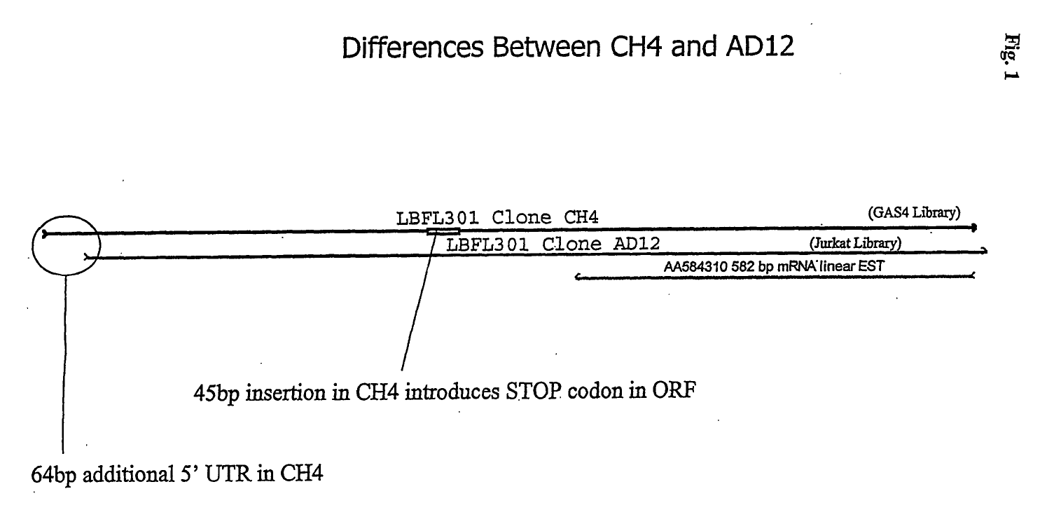

An open reading frame within the cDNA nucleotide sequence of SEQ ID NO: 3, at nucleotides 174-584 (174-587 including the stop codon), encodes a protein of 137 amino acids. The amino acid sequence corresponding to a predicted protein encoded by SEQ ID NO: 3 is set forth in SEQ ID NO: 4. The protein sequence of SEQ ID NO: 4 is identical to that of SEQ ID NO: 2 for the first 124 amino acids, while the last 13 amino acids of SEQ ID NO: 4 are unique. As shown in FIG. 1, termination of the protein sequence corresponding to SEQ ID NO: 4 is produced by a 45-bp insertion which introduces a stop codon in the open reading frame.

SEQ ID NOS: 2 and 4 are weakly similar to the chymotrypsin serine protease family signature (S1) and the NUDIX hydrolase family signature. The chymotrypsin serine protease family signature (S1) contains three domains, the third of which is absent in SEQ ID NO: 4. Additionally, both proteins contain a domain of collagen triple helix repeats.

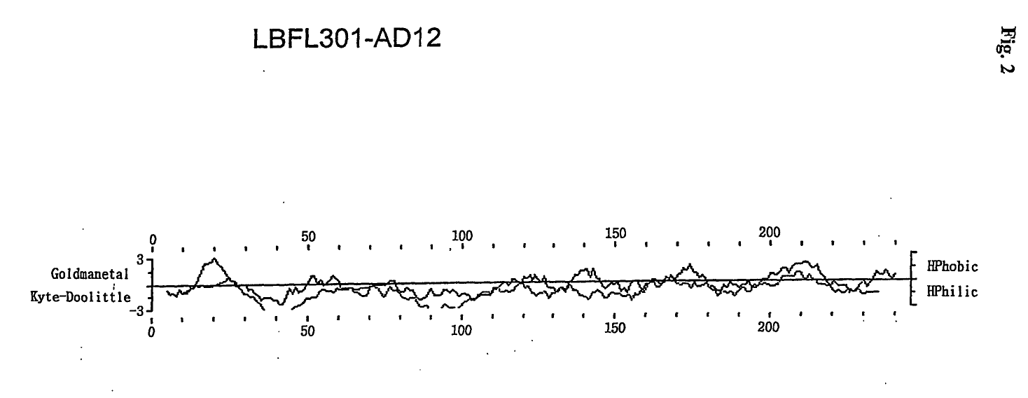

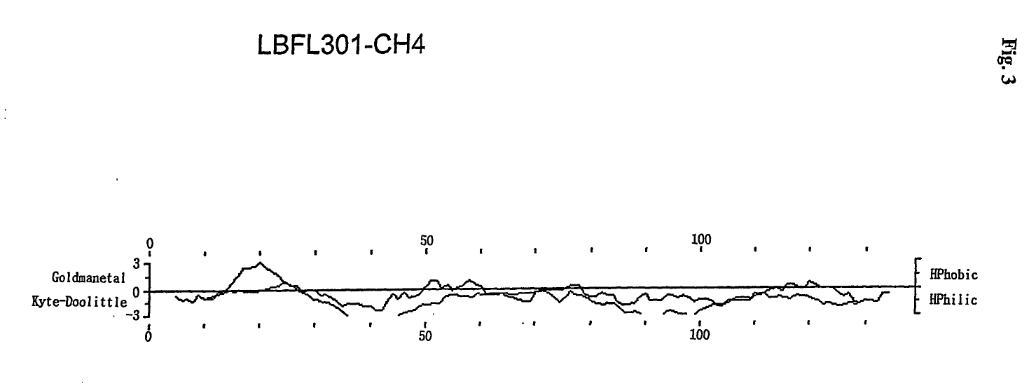

FIGS. 2 and 3 show the results of a hydrophobicity analysis of the amino acid sequence of SEQ ID NOS: 2 and 4. Hydrophilic regions may be used to produce antigenic peptides, as described above. Both sequences have hydrophobic N-termini, approximately 30 amino acids in length, with the most hydrophobic portion peaking at around amino acid no. 20. Further protein sequence-analysis by SPScan (GCG Wisconsin Package) reveals that the hydrophobic regions from amino acid positions 1-30 are likely to be secretory signal peptides.

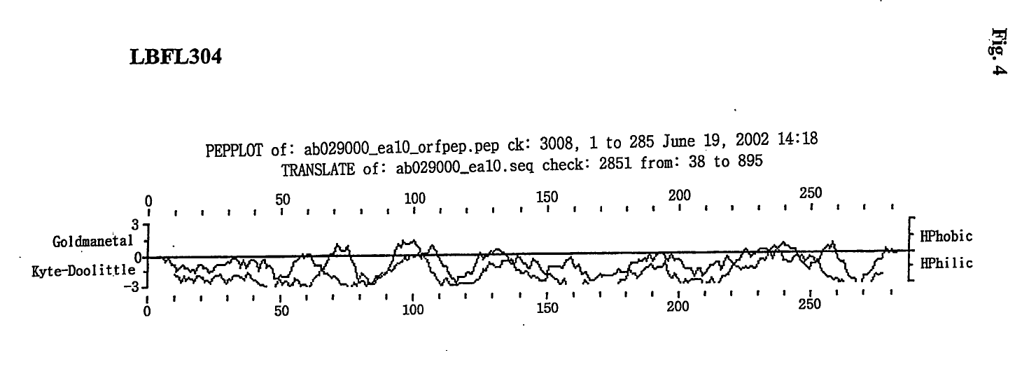

An open reading frame within the cDNA nucleotide sequence of SEQ ID NO: 5, at nucleotides 38-892 (38-895 including the stop codon), encodes a protein of 285 amino acids. The amino acid sequence corresponding to a predicted protein encoded by SEQ ID NO: 5 is set forth in SEQ ID NO: 6. SEQ ID NO: 6 is weakly similar to the chymotrypsin serine protease family (S1) signature. FIG. 4 shows the results of a hydrophobicity analysis of the amino acid sequence of SEQ ID NO: 6. Hydrophilic regions may be used to produce antigenic peptides, as described above.

An open reading frame within the cDNA nucleotide sequence of SEQ ID NO: 7, at nucleotides 53-892 (53-895 including the stop codon), encodes a protein of 280 amino acids. The amino acid sequence corresponding to a predicted protein encoded by SEQ ID NO: 7 is set forth in SEQ ID NO: 8. The protein sequence of SEQ ID NO: 8 is identical to that of SEQ ID NO: 6, except that SEQ ID NO: 8 lacks the first five amino acids at the N-terminus of SEQ ID NO: 6.

An open reading frame within the cDNA nucleotide sequence of SEQ ID NO: 9, at nucleotides 65-892 (65-895 including the stop codon), encodes a protein of 276 amino acids. The amino acid sequence corresponding to a predicted protein encoded by SEQ ID NO: 9 is set forth in SEQ ID NO: 10. The protein sequence of SEQ ID NO: 10 is identical to that of SEQ ID NO: 6, except that SEQ ID NO: 10 lacks the first nine amino acids at the N-terminus of SEQ ID NO: 6.

An open reading frame within the cDNA nucleotide sequence of SEQ ID NO: 11, at nucleotides 92-892 (92-895 including the stop codon), encodes a protein of 267 amino acids. The amino acid sequence corresponding to a predicted protein encoded by SEQ ID NO: 11 is set forth in SEQ ID NO: 12. The protein sequence of SEQ ID NO: 12 is identical to that of SEQ ID NO: 6, except that SEQ ID NO: 12 lacks the first 18 amino acids at the N-terminus of SEQ ID NO: 6.

An open reading frame within the cDNA nucleotide sequence of SEQ ID NO: 13, at nucleotides 49-1434 (49-1437 including the stop codon), encodes a protein of 462 amino acids. The amino acid sequence corresponding to the protein encoded by SEQ ID NO: 13 is set forth in SEQ ID NO: 14.

BLAST search results and a high level of homology between the two sequences suggest that LBFL305 is a splice variant of Mst1 (e.g., of SEQ ID NO: 16). The underlined amino acid residues of the alignment indicate the differences between SEQ ID NO: 14 and SEQ ID NO: 18. Based on published studies of Mst1, SEQ ID NO: 14 contains a kinase domain (amino acid positions 1-299) (Creasy et al., (1996) J Biol Chem 271:21049-21053), followed by a regulatory domain which acts to regulate kinase function (amino acid positions 300-462) (Creasy et al., (1996) J Biol Chem 271:21049-21053). Also present are two caspase cleavage sites, between amino acid positions 326-327 and 349-350 (Graves et al., (2001) J Biol Chem 276:14909-14915), and one NES domain (amino acid positions 361-370) (Ura et al., (2002) Proc Natl Acad Sci USA 98: 10148-10153). Compared to SEQ ID NO: 16, SEQ ID NO: 14 is missing the second NES domain (amino acid positions 441-451 in SEQ ID NO: 16) (Ura et al., (2002) Proc Natl Acad Sci USA 98: 10148-10153). Also, SEQ ID NO: 14 does not contain the multimerization domain (amino acid positions 431-487 in Mst1) that is required for self-association (Creasy et al., (1996) J Biol Chem 271:21049-21053). Interestingly, the region in Mst1 that is required for its interaction with NORE, a putative Ras effector (amino acid positions 449-487 in SEQ ID NO: 16) (Khokhlatchev et al., Curr Biol 12:253-265), is absent in SEQ ID NO: 14.

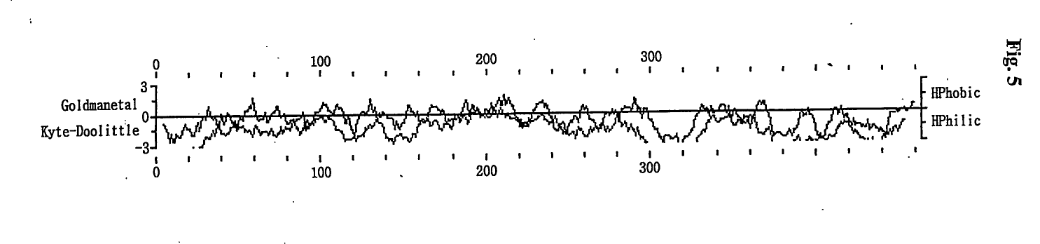

FIG. 5 show the results of a hydrophobicity analysis of the amino acid sequence of SEQ ID NO: 14. Hydrophilic regions may be used to produce antigenic peptides, as described above.

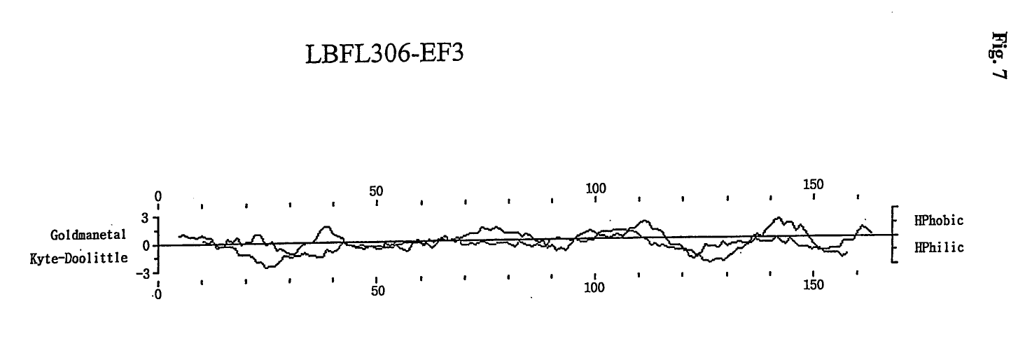

An open reading frame within the cDNA nucleotide sequence of SEQ ID NO: 17, at nucleotides 75-572 (75-575 including the stop codon), encodes a protein of 166 amino acids. The amino acid sequence corresponding to the protein encoded by SEQ ID NO: 17 is set forth in SEQ ID NO: 18. FIG. 7 shows the results of a hydrophobicity analysis of the amino acid sequence of SEQ ID NO: 18. Hydrophilic regions may be used to produce antigenic peptides, as described above.

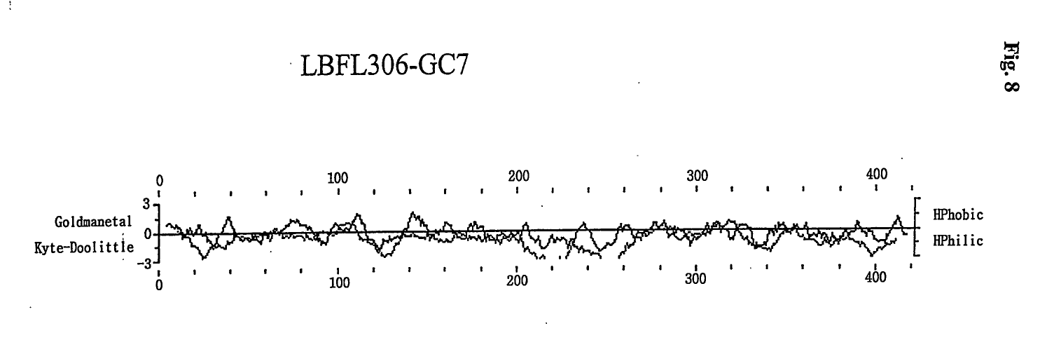

An open reading frame within the cDNA nucleotide sequence of SEQ ID NO: 19, at nucleotides 78-1337 (78-1340 including the stop codon), encodes a protein of 420 amino acids. The amino acid sequence corresponding to the protein encoded by SEQ ID NO: 19 is set forth in SEQ ID NO: 20. FIG. 8 shows the results of a hydrophobicity analysis of the amino acid sequence of SEQ ID NO: 20. Hydrophilic regions may be used to produce antigenic peptides, as described above.

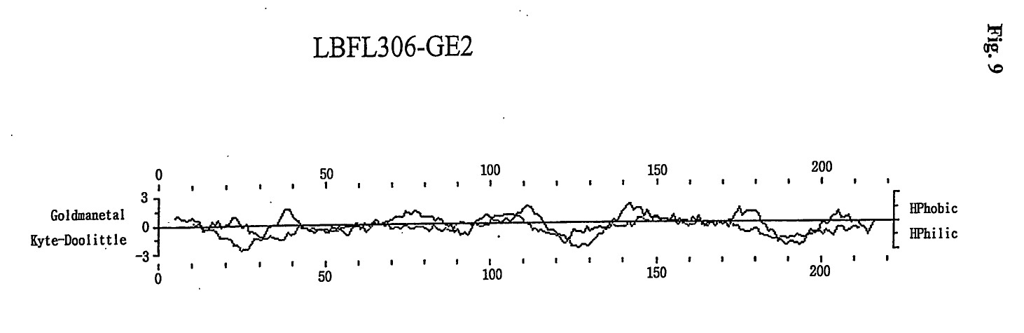

An open reading frame within the cDNA nucleotide sequence of SEQ ID NO: 19, at nucleotides 78-737 (78-740 including the stop codon), encodes a protein of 220 amino acids. The amino acid sequence corresponding to the protein encoded by SEQ ID NO: 21 is set forth in SEQ ID NO: 22. FIG. 9 shows the results of a hydrophobicity analysis of the amino acid sequence of SEQ ID NO: 22. Hydrophilic regions may be used to produce antigenic peptides, as described above.

All three LBFL306 clones, EF3 (SEQ ID NO: 17), GC7 (SEQ ID NO: 19) and GE2 (SEQ ID NO: 21), contain multiple ankyrin repeats, as determined by hmmerpfam, using GCG Wisconsin Package software. The ankyrin repeats are from amino acid residues 57 to 89, 91 to 123 and 124 to 156 in EF3, GC7 and GE2. In addition to these three ankyrin repeats, GC7 contains an additional ankyrin repeat from residues 157 to 190.

Analysis by Northern blot was performed to determine the size of the mRNA transcripts that correspond to LBFL301, LBFL304 and LBFL305. Northern blots containing total RNAs from various human tissues were used (ClonTech), and clone CH4 (SEQ ID NO: 3), clone EA10 (SEQ ID NO: 5, 7, 9 or 11) and LBFL305 (SEQ ID NO: 13) were radioactively labeled by the random primer method and used to probe the blots. The blots were hybridized in Church and Gilbert buffer at 65° C. and washed with 0.1×SSC containing 0.1% SDS at room temperature. The Northern blots show a single transcript for each gene, which is approximately 1.57 kb (BFL301), 2.6 kb (BFL304) and 7.95 kb (LBFL305) in size. These correspond to the sizes of the inserts in clone CH4 (1.355 kb), clone EA10 (SEQ ID NO: 5, 7, 9 or 11), and LBFL305(6.5 kb). When the sequence of clone AD 12 (SEQ ID NO: 1) was used as the probe, a transcript of 1.44 kb was detected, which corresponds to the size of the insert, 1.272 kb, in clone AD12.

To examine the expression of LBFL301, LBFL304, LBFL305 or LBFL306 in various normal tissues, an electronic Northern blot (e-Northern) was prepared as follows. Using the chips and the procedures in Example 1, mRNA from a panel of normal tissues, as listed in Table 3, was hybridized to Affymetrix U95 human GeneChips. The results of these experiments is shown in Table 3. For each tissue type, the number of samples that are called present or absent are indicated, together with the total number of samples in that sample set. In addition, the median value and the 25^{th} and 75^{th} percentiles in each tissue type are listed. Interestingly, although this gene is up-regulated in stomach cancer, expression of LBFL301 or LBFL304 could not be detected in most normal stomach samples. In addition, although LBFL305 and LBFL306 were found in most normal stomach samples tested, the level of expression was lower than in most other normal tissues tested. This observation indicates that LBFL301, LBFL304, LBFL305 or LBFL306 may be used as a diagnostic agent or marker to detect or screen for stomach cancer, as discussed below. Expression levels of LBFL301 appeared to be highest in skin tissue, followed by placental, adipose, arterial, bladder, bone, breast and soft tissues. Lower levels of expression were detected in most of the other tissues listed in Table 3a, although this gene was not detected in the liver or in most areas of the brain and heart. Expression levels of LBFL304 appeared to be highest in the arteries, omentum, uterus, endometrium, myometrium, and prostate. Expression levels of LBFL305 appeared to be highest in organs of the immune system (white blood cells, lymph nodes, spleen and thymus gland) followed by samples from the appendix, artery, bone and lung. Still lower levels of expression were detected in most of the other tissues listed in Table 3c. Expression levels of LBFL306 appeared to be highest in organs of the immune system (e.g., lymph nodes, spleen and thymus gland) and of the reproductive system (e.g., breast, endometrium, prostate and uterus).

TABLE 3a
e-Northern Data for 48774_at: LBFL301 Gene Expression in Normal Tissues

| Global | | | | | | |
|---|---|---|---|---|---|---|
| Present | | | | | | |
| Freq. | Tissue | Present | Absent | Lower 25% | Median | Upper 75% |
| 0.5492 | Adipose | 29 of 33 | 4 of 33 | 130.90 | 200.78 | 302.98 |
| | Adrenal Gland | 1 of 12 | 11 of 12 | −4.10 | 8.75 | 22.07 |
| | Appendix | 1 of 3 | 2 of 3 | 21.54 | 31.31 | 71.81 |
| | Artery | 3 of 3 | 0 of 3 | 148.46 | 203.96 | 262.32 |
| | Bladder | 6 of 7 | 1 of 7 | 142.72 | 195.44 | 361.02 |
| | Bone | 2 of 4 | 2 of 4 | 75.00 | 240.38 | 412.62 |
| | Breast | 74 of 82 | 8 of 82 | 104.43 | 222.98 | 506.27 |
| | Cerebellum | 0 of 5 | 5 of 5 | −7.51 | −6.91 | 0.94 |
| | Cervix | 75 of 99 | 24 of 99 | 42.04 | 93.36 | 144.25 |
| | Colon | 36 of 148 | 112 of 148 | 1.75 | 12.16 | 29.05 |
| | Cortex Frontal Lobe | 1 of 7 | 6 of 7 | 5.96 | 14.07 | 18.05 |
| | Cortex Temporal Lobe | 0 of 3 | 3 of 3 | −0.59 | 4.13 | 4.66 |
| | Duodenum | 9 of 61 | 52 of 61 | 3.99 | 11.54 | 20.62 |
| | Endometrium | 16 of 21 | 5 of 21 | 46.25 | 85.18 | 113.55 |
| | Esophagus | 14 of 27 | 13 of 27 | 17.91 | 42.08 | 81.58 |
| | Fallopian Tube | 21 of 51 | 30 of 51 | 7.97 | 20.39 | 33.96 |
| | GallBladder | 4 of 8 | 4 of 8 | 16.22 | 80.97 | 400.47 |
| | Heart | 0 of 3 | 3 of 3 | −3.80 | 6.00 | 11.57 |
| | Hippocampus | 1 of 5 | 4 of 5 | −6.49 | −0.18 | 7.81 |
| | Kidney | 12 of 87 | 75 of 87 | −14.80 | −4.61 | 9.24 |
| | Larynx | 4 of 4 | 0 of 4 | 48.51 | 119.88 | 248.62 |
| | Left Atrium | 64 of 141 | 77 of 141 | 8.19 | 27.15 | 61.94 |
| | Left Ventricle | 2 of 15 | 13 of 15 | −7.49 | 7.08 | 13.24 |
| | Liver | 0 of 33 | 33 of 33 | −15.13 | −8.62 | 0.03 |
| | Lung | 43 of 92 | 49 of 92 | 10.45 | 30.14 | 63.21 |
| | Lymph Node | 9 of 12 | 3 of 12 | 43.28 | 81.96 | 225.75 |
| | Muscles | 19 of 38 | 19 of 38 | 23.70 | 40.22 | 108.40 |
| | Myometrium | 68 of 106 | 38 of 106 | 19.39 | 56.42 | 99.78 |
| | Omentum | 12 of 16 | 4 of 16 | 76.26 | 148.41 | 236.54 |
| | Ovary | 26 of 75 | 49 of 75 | 4.20 | 21.96 | 47.43 |
| | Pancreas | 7 of 34 | 27 of 34 | −12.61 | 0.83 | 17.69 |
| | Placenta | 5 of 5 | 0 of 5 | 284.63 | 361.07 | 414.51 |
| | Prostate | 7 of 32 | 25 of 32 | 0.03 | 12.08 | 36.90 |
| | Rectum | 17 of 44 | 27 of 44 | 3.23 | 12.57 | 37.41 |
| | Right Atrium | 60 of 171 | 111 of 171 | 2.99 | 15.73 | 53.22 |
| | Right Ventricle | 43 of 160 | 117 of 160 | 1.85 | 16.64 | 39.58 |
| | Skin | 56 of 59 | 3 of 59 | 321.45 | 906.78 | 1515.60 |
| | Small Intestine | 18 of 68 | 50 of 68 | 0.41 | 12.19 | 28.53 |
| | Soft Tissues | 5 of 6 | 1 of 6 | 148.50 | 202.33 | 794.03 |
| | Spleen | 5 of 29 | 24 of 29 | −3.61 | 3.04 | 12.46 |
| | Stomach | 15 of 45 | 30 of 45 | 7.73 | 18.66 | 50.97 |
| | Testis | 3 of 5 | 2 of 5 | 14.11 | 27.34 | 64.24 |
| | Thymus | 19 of 71 | 52 of 71 | 4.06 | 25.61 | 40.45 |
| | Thyroid Gland | 7 of 19 | 12 of 19 | 12.43 | 32.64 | 40.31 |
| | Uterus | 35 of 56 | 21 of 56 | 32.20 | 44.73 | 143.10 |
| | WBC | 1 of 41 | 40 of 41 | −18.91 | −13.33 | −6.24 |

TABLE 3b
e-Northern for 35832_at: LBFL304 Gene Expression in Normal Tissues

| | Global | | | | | | |
|---|---|---|---|---|---|---|---|
| | Present | | | | Lower | | Upper |
| Fragment | Freq. | Tissue | Present | Absent | 25% | Median | 75% |
| 35832_at | 0.5228 | Adipose | 26 of 34 | 8 of 34 | 29.09 | 59.51 | 89.67 |
| | | Adrenal Gland | 1 of 12 | 11 of 12 | −11.00 | −6.08 | 8.14 |
| | | Appendix | 1 of 3 | 2 of 3 | 43.26 | 53.50 | 66.52 |
| | | Artery | 3 of 4 | 1 of 4 | 182.70 | 291.81 | 428.36 |
| | | Bladder | 5 of 7 | 2 of 7 | 56.36 | 62.71 | 64.68 |
| | | Bone | 3 of 4 | 1 of 4 | 19.34 | 77.40 | 167.06 |
| | | Breast | 65 of 82 | 17 of 82 | 33.67 | 63.19 | 108.61 |
| | | Cerebellum | 0 of 5 | 5 of 5 | −19.26 | −14.78 | −13.16 |
| | | Cervix | 69 of 102 | 33 of 102 | 18.76 | 57.45 | 94.99 |
| | | Colon | 85 of 146 | 61 of 146 | 10.73 | 35.22 | 87.91 |
| | | Cortex Frontal | 1 of 7 | 6 of 7 | −5.03 | 8.78 | 14.71 |
| | | Lobe | | | | | |
| | | Cortex | 0 of 3 | 3 of 3 | −16.73 | −16.67 | −15.85 |
| | | Temporal Lobe | | | | | |
| | | Duodenum | 19 of 53 | 34 of 53 | 6.47 | 20.39 | 41.95 |
| | | Endometrium | 15 of 21 | 6 of 21 | 31.44 | 93.20 | 137.68 |
| | | Esophagus | 15 of 27 | 12 of 27 | 5.12 | 27.03 | 52.89 |
| | | Fallopian Tube | 19 of 47 | 28 of 47 | 5.38 | 22.48 | 54.99 |
| | | GallBladder | 2 of 7 | 5 of 7 | 8.71 | 28.94 | 50.85 |
| | | Heart | 0 of 3 | 3 of 3 | −35.98 | −28.25 | −6.72 |
| | | Hippocampus | 2 of 5 | 3 of 5 | −7.43 | −3.64 | 5.68 |
| | | Kidney | 28 of 89 | 61 of 89 | 1.67 | 20.45 | 45.18 |
| | | Larynx | 4 of 4 | 0 of 4 | 36.13 | 54.20 | 79.75 |
| | | Left Atrium | 80 of 141 | 61 of 141 | 8.32 | 25.37 | 52.28 |
| | | Left Ventricle | 0 of 15 | 15 of 15 | −21.85 | −17.01 | −8.17 |
| | | Liver | 2 of 35 | 33 of 35 | −10.51 | 0.02 | 8.05 |
| | | Lung | 29 of 93 | 64 of 93 | 2.56 | 19.47 | 43.63 |
| | | Lymph Node | 3 of 12 | 9 of 12 | −17.58 | −2.85 | 9.56 |
| | | Muscles | 12 of 42 | 30 of 42 | −13.74 | 3.99 | 23.23 |
| | | Myometrium | 92 of 108 | 16 of 108 | 67.57 | 129.39 | 203.58 |
| | | Omentum | 14 of 15 | 1 of 15 | 176.65 | 310.28 | 368.41 |
| | | Ovary | 31 of 74 | 43 of 74 | 0.66 | 27.78 | 54.33 |
| | | Pancreas | 4 of 33 | 29 of 33 | −9.60 | 2.09 | 9.94 |
| | | Placenta | 0 of 5 | 5 of 5 | −21.32 | −3.06 | 6.08 |
| | | Prostate | 30 of 32 | 2 of 32 | 82.37 | 104.56 | 190.72 |
| | | Rectum | 37 of 44 | 7 of 44 | 51.53 | 86.73 | 125.67 |
| | | Right Atrium | 69 of 170 | 101 of 170 | −3.30 | 8.80 | 33.56 |
| | | Right Ventricle | 35 of 160 | 125 of 160 | −11.65 | −0.46 | 16.02 |
| | | Skin | 28 of 61 | 33 of 61 | 4.22 | 25.33 | 67.56 |
| | | Small Intestine | 36 of 67 | 31 of 67 | 10.76 | 33.92 | 64.75 |
| | | Soft Tissues | 4 of 6 | 2 of 6 | 25.95 | 40.91 | 58.70 |
| | | Spleen | 1 of 29 | 28 of 29 | −19.20 | −13.77 | −6.69 |
| | | Stomach | 16 of 47 | 31 of 47 | −8.30 | 13.38 | 47.93 |
| | | Testis | 1 of 5 | 4 of 5 | −18.20 | 5.01 | 37.66 |
| | | Thymus | 1 of 73 | 72 of 73 | −22.55 | −12.50 | −3.27 |
| | | Thyroid Gland | 14 of 19 | 5 of 19 | 45.56 | 98.30 | 141.24 |
| | | Uterus | 43 of 58 | 15 of 58 | 37.47 | 103.26 | 180.98 |
| | | WBC | 0 of 43 | 43 of 43 | −33.45 | −25.32 | −20.23 |

TABLE 3c
e-Northern Data for 48774_at: LBFL305 Gene Expression in Normal Tissues

| Global | | | | | | |
|---|---|---|---|---|---|---|
| Present | | | | | | |
| Freq. | Tissue | Present | Absent | Lower 25% | Median | Upper 75% |
| 0.9444 | Adipose | 31 of 32 | 1 of 32 | 221.82 | 286.63 | 380.88 |
| | Adrenal Gland | 12 of 12 | 0 of 12 | 162.12 | 214.21 | 310.82 |
| | Appendix | 3 of 3 | 0 of 3 | 352.94 | 506.01 | 633.71 |
| | Artery | 3 of 3 | 0 of 3 | 343.80 | 419.88 | 643.55 |
| | Bladder | 5 of 5 | 0 of 5 | 221.82 | 290.82 | 301.11 |
| | Bone | 3 of 3 | 0 of 3 | 410.63 | 508.38 | 662.78 |
| | Breast | 80 of 80 | 0 of 80 | 236.84 | 279.81 | 338.22 |
| | Cerebellum | 5 of 5 | 0 of 5 | 182.09 | 198.28 | 283.55 |
| | Cervix | 97 of 101 | 4 of 101 | 179.11 | 246.50 | 317.62 |
| | Colon | 146 of 151 | 5 of 151 | 247.18 | 314.49 | 389.23 |
| | Cortex Frontal Lobe | 7 of 7 | 0 of 7 | 222.19 | 230.28 | 268.13 |
| | Cortex Temporal Lobe | 3 of 3 | 0 of 3 | 305.66 | 365.62 | 377.16 |
| | Duodenum | 58 of 61 | 3 of 61 | 206.17 | 276.14 | 331.91 |
| | Endometrium | 21 of 21 | 0 of 21 | 158.91 | 193.40 | 257.17 |
| | Esophagus | 25 of 27 | 2 of 27 | 182.29 | 223.24 | 303.93 |
| | Eallopian Tube | 50 of 51 | 1 of 51 | 168.69 | 220.72 | 265.95 |
| | GallBladder | 7 of 8 | 1 of 8 | 237.67 | 270.08 | 312.45 |
| | Heart | 2 of 3 | 1 of 3 | 44.79 | 55.84 | 56.46 |
| | Hippocampus | 5 of 5 | 0 of 5 | 165.94 | 212.72 | 328.59 |
| | Kidney | 79 of 86 | 7 of 86 | 121.83 | 158.99 | 209.67 |
| | Larynx | 4 of 4 | 0 of 4 | 140.76 | 209.46 | 302.84 |
| | Left Atrium | 127 of 141 | 14 of 141 | 58.48 | 92.78 | 123.06 |
| | Left Ventricle | 9 of 15 | 6 of 15 | 50.50 | 74.69 | 101.06 |
| | Liver | 27 of 34 | 7 of 34 | 87.73 | 146.60 | 197.27 |
| | Lung | 92 of 93 | 1 of 93 | 365.58 | 454.87 | 550.48 |
| | Lymph Node | 11 of 11 | 0 of 11 | 493.34 | 943.95 | 1141.06 |
| | Muscles | 19 of 39 | 20 of 39 | 41.41 | 64.44 | 110.74 |
| | Myometrium | 104 of 106 | 2 of 106 | 188.94 | 263.73 | 322.65 |
| | Omentum | 15 of 15 | 0 of 15 | 198.02 | 244.56 | 334.75 |
| | Ovary | 70 of 74 | 4 of 74 | 133.44 | 181.39 | 246.40 |
| | Pancreas | 13 of 34 | 21 of 34 | 24.30 | 53.13 | 84.64 |
| | Placenta | 4 of 5 | 1 of 5 | 156.58 | 174.21 | 182.71 |
| | Prostate | 32 of 32 | 0 of 32 | 184.16 | 234.60 | 289.05 |
| | Rectum | 42 of 43 | 1 of 43 | 284.60 | 365.66 | 434.01 |
| | Right Atrium | 148 of 169 | 21 of 169 | 54.96 | 89.92 | 129.13 |
| | Right Ventricle | 132 of 160 | 28 of 160 | 55.58 | 78.85 | 114.70 |
| | Skin | 57 of 59 | 2 of 59 | 250.81 | 320.57 | 398.56 |
| | Small Intestine | 64 of 68 | 4 of 68 | 196.50 | 279.29 | 393.83 |
| | Soft Tissues | 6 of 6 | 0 of 6 | 234.21 | 307.07 | 363.66 |
| | Spleen | 31 of 31 | 0 of 31 | 775.25 | 879.84 | 1022.49 |
| | Stomach | 41 of 47 | 6 of 47 | 137.91 | 217.53 | 338.54 |
| | Testis | 5 of 5 | 0 of 5 | 326.62 | 358.69 | 377.26 |
| | Thymus | 71 of 71 | 0 of 71 | 691.12 | 802.96 | 984.42 |
| | Thyroid Gland | 18 of 18 | 0 of 18 | 121.11 | 162.16 | 238.53 |
| | Uterus | 57 of 58 | 1 of 58 | 157.19 | 202.53 | 265.91 |
| | WBC | 38 of 40 | 2 of 40 | 1863.06 | 2264.27 | 2743.82 |

TABLE 3d
e-Northern Data for 48774_at: LBFL306 Gene Expression in Normal Tissues

| Global | | | | Lower | | Upper |
|---|---|---|---|---|---|---|
| Present Freq. | Tissue | Present | Absent | 25% | Median | 75% |
| 0.8143 | Adipose | 31 of 32 | 1 of 32 | 184.04 | 242.67 | 285.20 |
| | Adrenal Gland | 6 of 12 | 6 of 12 | 130.70 | 157.08 | 187.26 |
| | Appendix | 3 of 3 | 0 of 3 | 259.39 | 301.05 | 388.31 |
| | Artery | 4 of 4 | 0 of 4 | 168.95 | 207.06 | 295.97 |
| | Bladder | 7 of 8 | 1 of 8 | 196.52 | 239.43 | 374.54 |
| | Bones | 4 of 4 | 0 of 4 | 209.35 | 226.22 | 292.55 |
| | Breast | 60 of 61 | 1 of 61 | 238.09 | 315.29 | 421.47 |
| | Cervix | 92 of 102 | 10 of 102 | 180.90 | 224.31 | 342.25 |
| | Colon | 168 of 192 | 24 of 192 | 147.41 | 175.83 | 215.95 |
| | Cortex Frontal Lobe | 5 of 5 | 0 of 5 | 133.94 | 148.22 | 162.91 |
| | Cortex Temporal Lobe | 3 of 3 | 0 of 3 | 137.28 | 147.77 | 172.44 |
| | Duodenum | 68 of 68 | 0 of 68 | 186.24 | 218.73 | 336.00 |
| | Endometrium | 19 of 19 | 0 of 19 | 273.57 | 359.17 | 436.85 |
| | Esophagus | 14 of 25 | 11 of 25 | 105.83 | 140.56 | 195.89 |
| | Gall Bladder | 7 of 8 | 1 of 8 | 232.71 | 293.00 | 410.47 |
| | Heart | 1 of 3 | 2 of 3 | 86.39 | 100.48 | 129.68 |
| | Hippocampus | 9 of 10 | 1 of 10 | 125.62 | 140.94 | 194.06 |
| | Kidney | 53 of 91 | 38 of 91 | 108.29 | 147.35 | 186.69 |
| | Larynx | 3 of 4 | 1 of 4 | 160.96 | 190.50 | 219.55 |
| | Left Atrium | 65 of 143 | 78 of 143 | 100.94 | 128.86 | 157.57 |
| | Left Ventricle | 0 of 13 | 13 of 13 | 82.54 | 106.63 | 117.06 |
| | Liver | 15 of 44 | 29 of 44 | 145.82 | 204.77 | 244.58 |
| | Lung | 104 of 114 | 10 of 114 | 168.03 | 203.35 | 283.11 |
| | Lymph Node | 14 of 14 | 0 of 14 | 207.66 | 319.53 | 366.70 |
| | Lymphocytes(B + T Cells) | 24 of 24 | 0 of 24 | 224.46 | 292.01 | 348.07 |
| | Muscles | 31 of 40 | 9 of 40 | 183.43 | 240.15 | 329.99 |
| | Myometrium | 122 of 128 | 6 of 128 | 209.83 | 244.58 | 294.20 |
| | Omentum | 13 of 15 | 2 of 15 | 162.83 | 236.23 | 265.24 |
| | Ovary | 80 of 81 | 1 of 81 | 219.28 | 259.00 | 331.64 |
| | Pancreas | 8 of 40 | 32 of 40 | 97.07 | 136.80 | 174.54 |
| | Prostate | 47 of 47 | 0 of 47 | 318.31 | 397.81 | 525.43 |
| | Rectum | 38 of 46 | 8 of 46 | 143.79 | 188.95 | 232.42 |
| | Right Atrium | 87 of 162 | 75 of 162 | 104.22 | 132.56 | 161.54 |
| | Skin | 38 of 44 | 6 of 44 | 162.29 | 198.90 | 236.86 |
| | Small Intestine | 72 of 79 | 7 of 79 | 184.41 | 230.96 | 270.34 |
| | Soft Tissues | 5 of 5 | 0 of 5 | 240.30 | 258.33 | 499.21 |
| | Spleen | 36 of 36 | 0 of 36 | 253.03 | 322.87 | 390.61 |
| | Stomach | 32 of 54 | 22 of 54 | 139.62 | 174.76 | 239.47 |
| | Thymus | 70 of 70 | 0 of 70 | 352.12 | 438.94 | 511.11 |
| | Thyroid Gland | 25 of 25 | 0 of 25 | 177.32 | 211.27 | 276.25 |
| | Uterus | 54 of 56 | 2 of 56 | 243.74 | 312.71 | 387.50 |
| | WBC | 21 of 25 | 4 of 25 | 149.63 | 176.72 | 209.49 |

### INDUSTRIAL APPLICAVILITY

### Example 3

Detection of LBFL301, LBFL304, LBFL305 or LBFL306 mRNA for Stomach Cancer Screening

The expression level of mRNA corresponding to SEQ ID NO: 1, 3, 5, 7, 9, 11, 13, 17, 19 or 21 is determined in stomach tissue biopsy samples, as described in Example 1, i.e., by screening mRNA samples on a GeneChip, or as described in Example 2, i.e., by screening mRNA samples on a Northern blot. Alternatively, samples from non-stomach hyperplastic tissues in malignant or non-malignant states may also be analyzed. Stomach tissue samples from patients with stomach cancer and from normal subjects may be used as positive and negative controls. Using any means of analyzing gene expression, a level of expression higher than that of the normal control is indicative of stomach cancer or a likelihood of developing stomach cancer.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1. An isolated nucleic acid molecule selected from the group consisting of: (a) an isolated nucleic acid molecule comprising SEQ ID NO: 3, 5, 7, 9, 11, 13, 17 or 19; (b) an isolated nucleic acid molecule that encodes the amino acid sequence of SEQ ID NO: 4, 14 or 18; (c) an isolated nucleic acid molecule that encodes a protein that is expressed in stomach cancer and that exhibits at least about 92% nucleotide sequence identity over the entire length of SEQ ID NO: 3 or 17; (d) an isolated nucleic acid molecule that encodes a protein that is expressed in stomach cancer and that exhibits at least about 95% nucleotide sequence identity over the entire length of SEQ ID NO: 13; (e) an isolated nucleic acid molecule comprising the complement of a nucleic acid molecule of (a), (b), (c) or (d).

2. The isolated nucleic acid molecule of claim 1, wherein the nucleic acid molecule comprises nucleotides 174-584 of SEQ ID NO: 3.

3. The isolated nucleic acid molecule of claim 1, wherein the nucleic acid molecule consists of nucleotides 174-584 of SEQ ID NO: 3.

4. The isolated nucleic acid molecule of claim 1, wherein the nucleic acid molecule comprises nucleotides 174-587 of SEQ ID NO: 3.

5. The isolated nucleic acid molecule of claim 1, wherein the nucleic acid molecule is selected from the group consisting of: a nucleic acid molecule consisting of nucleotides 38-892 of SEQ ID NO: 5 and a nucleic acid molecule consisting of nucleotides 38-895 of SEQ ID NO: 5.

6. The isolated nucleic acid molecule of claim 1, wherein the nucleic acid molecule is selected from the group consisting of a nucleic acid molecule consisting of nucleotides 53-892 of SEQ ID NO: 7 and a nucleic acid molecule consisting of nucleotides 53-895 of SEQ ID NO: 7.

7. The isolated nucleic acid molecule of claim 1, wherein the nucleic acid molecule is selected from the group consisting of: a nucleic acid molecule consisting of nucleotides 65-892 of SEQ ID NO: 9 and a nucleic acid molecule consisting of nucleotides 65-895 of SEQ ID NO: 9.

8. The isolated nucleic acid molecule of claim 1, wherein the nucleic acid molecule is selected from the group consisting of: a nucleic acid molecule consisting of nucleotides 92-892 of SEQ ID NO: 11 and a nucleic acid molecule consisting of nucleotides 92-895 of SEQ ID NO: 11.

9. The isolated nucleic acid molecule of claim 1, wherein the nucleic acid molecule comprises nucleotides 49-1434 of SEQ ID NO: 13.

10. The isolated nucleic acid molecule of claim 1, wherein the nucleic acid molecule consists of nucleotides 49-1437 of SEQ ID NO: 13.

11. The isolated nucleic acid molecule of claim 1, wherein the nucleic acid molecule comprises nucleotides 49-1437 of SEQ ID NO: 13.

12. The isolated nucleic acid molecule of claim 1, wherein the nucleic acid molecule comprises nucleotides 75-575 of SEQ ID NO: 17.

13. The isolated nucleic acid molecule of claim 1, wherein the nucleic acid molecule consists of nucleotides 75-575 of SEQ ID NO: 17.

14. The isolated nucleic acid molecule of claim 1, wherein the nucleic acid molecule comprises nucleotides 75-572 of SEQ ID NO: 17.

15. The isolated nucleic acid molecule of claim 1, wherein said nucleic acid molecule is operably linked to one or more expression control elements.

16. A vector comprising an isolated nucleic acid molecule of claim 1.

17. A host cell transformed to contain the nucleic acid molecule of claim 1.

18. A host cell comprising a vector of claim 16.

19. A host cell of claim 18, wherein said host cell is selected from the group consisting of prokaryotic host cells and eukaryotic host cells.

20. A method for producing a polypeptide or protein comprising culturing a host cell transformed with the nucleic acid molecule of claim 1 under conditions in which the polypeptide or protein encoded by said nucleic acid molecule is expressed.

21. The method of claim 20, wherein said host cell is selected from the group consisting of prokaryotic host cells and eukaryotic host cells.

22. An isolated polypeptide or protein produced by the method of claim 21.

23. An isolated polypeptide or protein selected from the group consisting of: (a) an isolated polypeptide or protein comprising the amino acid sequence of SEQ ID NO: 4, 6, 8, 10, 12, 14 or 18; (b) an isolated polypeptide or protein exhibiting at least about 92% amino acid sequence identity with SEQ ID NO: 4; (c) an isolated polypeptide or protein consisting of amino acids 31-137 of SEQ ID NO: 4; (d) an isolated polypeptide comprising a fragment of at least 10 amino acids of SEQ ID NO: 6, 8, 10 or 12; (e) an isolated polypeptide comprising conservative amino acid substitutions of SEQ ID NO: 6, 8, 10 or 12; (f) an isolated polypeptide comprising naturally occurring amino acid sequence variants of SEQ ID NO: 6, 8, 10 or 12; (g) an isolated polypeptide exhibiting at least about 75% amino acid sequence identity with SEQ ID NO: 6, 8, 10 or 12; (h) an isolated polypeptide or protein exhibiting at least about 95% amino acid sequence identity with SEQ ID NO: 14; and (i) an isolated polypeptide or protein exhibiting at least about 92% amino acid sequence identity with SEQ ID NO: 18.

24. An isolated antibody or antigen-binding antibody fragment that binds to a polypeptide or protein of claim 23 or to an isolated polypeptide or protein comprising the amino acid sequence of SEQ ID NO: 2.

25. An antibody of claim 24 wherein said antibody is a monoclonal or a polyclonal antibody.

26. A method of identifying an agent which modulates the expression of a nucleic acid encoding a protein of claim 23, a protein comprising the amino acid sequence of SEQ ID NO: 2, 20 or 22, or a Mst1 protein or a Mst1 splice variant protein, the method comprising:
exposing cells which express the nucleic acid to the agent; and
determining whether the agent modulates expression of said nucleic acid, thereby identifying an agent which modulates the expression of a nucleic acid encoding the protein.

27. A method of identifying an agent which modulates the level of or at least one activity of a protein of claim 23, or of a protein comprising the amino acid sequence of SEQ ID NO: 2, 20 or 22, or of a Mst1 protein or a Mst1 splice variant protein, the method comprising:
exposing cells which express the protein to the agent;
determining whether the agent modulates the level of or at least one activity of said protein, thereby identifying an agent which modulates the level of or at least one activity of the protein.

28. The method of claim 27, wherein the agent modulates one activity of the protein.

29. A method of identifying binding partners for a protein of claim 23 or a protein comprising the amino acid sequence of SEQ ID NO: 2, the method comprising:
exposing said protein to a potential binding partner; and
determining if the potential binding partner binds to said protein, thereby identifying binding partners for the protein.

30. A method of modulating the expression of a nucleic acid encoding a protein of claim 23, a protein comprising the amino acid sequence of SEQ ID NO: 2, 20 or 22, or a Mst1 protein or a Mst1 splice variant protein, the method comprising:
administering an effective amount of an agent which modulates the expression of a nucleic acid encoding the protein.

31. A method of modulating at least one activity of a protein of claim 23, or of a protein comprising the amino acid sequence of SEQ ID NO: 2, 20 or 22, or of a Mst1 protein or a Mst1 splice variant protein, the method comprising:
administering an effective amount of an agent which modulates at least one activity of the protein.

32. A non-human transgenic animal modified to contain a nucleic acid molecule of claim 1.

33. The transgenic animal of claim 32, wherein the nucleic acid molecule contains a mutation that prevents expression of the encoded protein.

34. A method of diagnosing a disease state in a subject, comprising:
determining the level of expression of a nucleic acid molecule of claim 1, or a nucleic acid molecule encoding a Mst1 protein or a Mst1 splice variant protein, or of a nucleic acid molecule encoding an isolated polypeptide or protein selected from the group consisting of: (a) an isolated polypeptide or protein comprising the amino acid sequence of SEQ ID NO: 4, 6, 8, 10, 12, 14 or 18; (b) an isolated polypeptide or protein exhibiting at least about 92% amino acid sequence identity with SEQ ID NO: 4; (c) an isolated polypeptide or protein consisting of amino acids 31-137 of SEQ ID NO: 4; (d) an isolated polypeptide comprising a fragment of at least 10 amino acids of SEQ ID NO: 6, 8, 10 or 12; (e) an isolated polypeptide comprising conservative amino acid substitutions of SEQ ID NO: 6, 8, 10 or 12; (f) an isolated polypeptide comprising naturally occurring amino acid sequence variants of SEQ ID NO: 6, 8, 10 or 12; (g) an isolated polypeptide exhibiting at least about 75% amino acid sequence identity with SEQ ID NO: 6, 8, 10 or 12; (h) an isolated polypeptide or protein exhibiting at least about 95% amino acid sequence identity with SEQ ID NO: 14; and (i) an isolated polypeptide or protein exhibiting at least about 92% amino acid sequence identity with SEQ ID NO: 18, or of a protein comprising the amino acid sequence of SEQ ID NO: 2, 20 or 22 or of a Mst1 protein or a Mst1 splice variant protein.

35. The method of claim 34, wherein the disease state is stomach cancer.

36. The method of claim 34, wherein the disease state is advanced gastric cancer.

37. The method of claim 34, wherein the disease state is a malignant neoplasm.

38. The method of claim 37, wherein the malignant neoplasm occurs in soft tissue, bone, breast, cervix, colon, endometrium, esophagus, kidney, larynx, liver, lung, omentum, ovary, pancreas, rectum, thyroid, myometrium, prostate, skin, small intestine, bladder, spleen or stomach.

39. The method of claim 20, wherein the splice variant is SEQ ID NO: 13 or SEQ ID NO: 14.

40. A composition comprising a diluent and a polypeptide or protein selected from the group consisting of: (a) an isolated polypeptide or protein comprising the amino acid sequence of SEQ ID NO: 4, 6, 8, 10, 12, 14, 18, 20 or 22 (b) an isolated polypeptide or protein exhibiting at least about 92% amino acid sequence identity with SEQ ID NO: 4 or 18, (c) an isolated polypeptide or protein consisting of amino acids 31-137 of SEQ ID NO: 4, (d) an isolated polypeptide comprising a fragment of at least 10 amino acids of SEQ ID NO: 6, 8, 10 or 12, (e) an isolated polypeptide comprising conservative amino acid substitutions of SEQ ID NO: 6, 8, 10 or 12, (f) an isolated polypeptide comprising naturally occurring amino acid sequence variants of SEQ ID NO: 6, 8, 10 or 12, (g) an isolated polypeptide exhibiting at least about 75% amino acid sequence identity with SEQ ID NO: 6, 8, 10 or 12, (h) an isolated polypeptide exhibiting at least about 95% amino acid sequence identity with SEQ ID NO: 14.
